(19) United States
(12) Patent Application Publication
(10) Pub. No.: US 2010/0285540 A1
(43) Pub. Date: Nov. 11, 2010

(54) Process for the preparation of (R)-Beta-Arylalanines by coupled Racemase/Aminomutase catalysis

(75) Inventors: Kevin D. Walker, Holt, MI (US); Brad M. Cox, Fortville, IN (US)

Correspondence Address: BUTZEL LONG, 2190 Commons Parkway, Okemos, MI, 48864, US

(73) Assignee: Board of Trustees of Michigan State University, East Lansing, MI (US)

(21) Appl. No.: 12/799,807

(22) Filed: Apr. 30, 2010

Related U.S. Application Data
(60) Provisional application No. 61/215,053, filed on May 1, 2009.

(51) Int. Cl. C12P 13/04; C12N 9/90; C12N 1/21; C12N 15/63; C07H 21/04

(52) U.S. Cl. 435/106; 435/233; 435/252.33; 435/320.1; 536/23.2; 536/24.33

(57) ABSTRACT

A process for producing (R)-β-arylalanines is described. The process converts (S)-α-arylalanine to (R)-β-arylalanine using phenylalanine aminomutase and amino acid racemase. The (R)-β-arylalanine product can be used to form pharmaceutically useful drugs. Also disclosed are DNA sequences encloding phenylalanine aminomutase and amino acid racemase enzymes, sequences for PCR primers of the DNA sequences, and sequences for expressed aminomutase and amino acid racemase enzymes.

### CROSS-REFERENCE TO RELATED APPLICATIONS

This application claims priority to Provisional Application No. 61/215,053, filed May 1, 2009, which is incorporated herein by reference in its entirety.

### STATEMENT REGARDING FEDERALLY SPONSORED RESEARCH OR DEVELOPMENT

This work was supported by the National Science Foundation (CAREER Award 0746432).

### REFERENCE TO A “COMPUTER LISTING APPENDIX SUBMITTED ON A COMPACT DISC”

The application contains nucleotide and amino acid sequences which are identified with SEQ ID NOS. A compact disc is provided which contains the Sequence Listings for the sequences. The Sequence Listing on the compact disc is identical to the paper copy of the Sequence Listing provided with the application.

### BACKGROUND OF THE INVENTION

(1) Field of the Invention

The present invention relates to an enzyme-mediated, dynamic kinetic resolution of racemic α-arylalanines to (R)-β-arylalanines. In particular, the present invention relates to the use of an amino acid racemase to maintain a 1:1 equilibrium between (S)-α-arylalanines and (R)-α-arylalanines, and a phenylalanine aminomutase to convert the (S)-α-arylalanine to the (R)-β-arylalanine, the resolution step.

(2) Description of Related Art

Enantiomerically pure β-amino acids are emerging as an important class of compounds due to the frequent occurrence of β-amino acid substructure motifs in pharmaceutically important natural products.¹ In addition, single β-arylalanines have shown anti-epileptogenesis activity,² while other optically active β-amino acids serve as useful chiral scaffolds for the synthesis of β-peptides, β-lactams, and biologically active natural products.³ Therefore, efficient methods for the production of enantiopure β-amino acids would benefit novel drug synthesis and development.

The phenylalanine aminomutase (PAM), isolated from Taxus plants, catalyzes the stereospecific isomerization of (S)-α-phenylalanine to a single isomeric product (R)-β-phenylalanine.⁴ In the host plant, the latter is the biosynthetic precursor of the phenylisoserine side chain of the antineoplastic drug paclitaxel (Taxol), which has application in the treatment of heart disease⁵⁻⁷ and cancer.⁸⁻¹⁰ In vitro studies showed that heterologously expressed PAM established an equilibrium constant that slightly favors the formation of the β-phenylalanine product over the substrate.¹¹ Moreover, the substrate specificity of PAM was remarkably flexible as demonstrated by the conversion of a homologous series of non-natural 2′-, 3′-, or 4′-substituted (S)-α-arylalanines, (S)-β-styryl-α-alanine, and (S)-β-heterole-α-alanines to their corresponding (R)-β-amino isomers by the aminomutase.⁴

Since PAM is specific for the (S)-enantiomer of the aryl α-amino acid substrates,¹² the proportion of (S)-α-arylalanine to (R)-β-arylalanine at equilibrium in a PAM-catalyzed reaction, in which the substrate is racemic α-arylalanine, is theoretically limited by the equilibrium constant between the (S)-α- and (R)-β-isomers; consequently, the non-productive (R)-α-arylalanine isomer accumulates.

### OBJECTS

It is an object of the present invention to provide an improved enzymatic process for producing (R)-β-arylalanines. Further, it is an object of the invention to provide a novel combination of enzyme catalysts produced by recombinant microorganisms. Further still, it is an object to provide a novel DNA for producing the enzymes in the microorganisms. These and other objects will become increasingly apparent by reference to the following description and the Figures.

### SUMMARY OF THE INVENTION

The present invention provides E. coli cells comprising a transformed plasmid encoding phenylalanine aminomutase deposited at Michigan State University (MSU) as MSU_pamec20080804. Further, the present invention provides a recombinant microorganism comprising a transformed plasmid encoding phenylalanine aminomutase, the plasmid comprising DNA (i) being optimized for expression in the microorganism with one or more codons modified in the 3′-base wobble position of the codon to be recognizable by tRNA in the microorganism and/or (ii) having at least 80% homology with DNA as set forth in SEQ ID NO: 1, excluding the 3′-base wobble position of any codon of the DNA. Further still, the present invention provides DNA primers for PCR amplification of DNA for encoding phenylalanine aminomutase as set forth in SEQ ID NO: 2 and SEQ ID NO: 3. Still further, the present invention provides a plasmid comprising a DNA encoding phenylalanine aminomutase, the DNA (i) being optimized for expression in a target microorganism with one or more codons modified in the 3′-base wobble position of the codon to be recognizable by tRNA in the target microorganism and/or (ii) having at least 80% homology with DNA as set forth in SEQ ID NO: 1, excluding the 3′-base wobble position of any codon of the DNA. Further, the present invention provides a DNA encoding phenylalanine aminomutase, the DNA (i) being optimized for expression in a target microorganism with one or more codons modified in the 3′-base wobble position of the codon to be recognizable by tRNA in the target microorganism and/or (ii) having at least 80% homology with DNA as set forth in SEQ ID NO: 1, excluding the 3′-base wobble position of any codon of the DNA. Further still, the present invention provides E. coli cells comprising a transformed plasmid encoding an amino acid racemase deposited at Michigan State University (MSU) as MSU_racec20080826. Still further, the present invention provides a recombinant microorganism comprising a transformed plasmid encoding amino acid racemase, the plasmid comprising DNA (i) being optimized for expression in the microorganism with one or more codons modified in the 3′-base wobble position of the codon to be recognizable by tRNA in the microorganism and/or (ii) having at least 80% homology with DNA set forth in SEQ ID NO: 4, excluding the 3′-base wobble position of any codon of the DNA. Further still, the present invention provides DNA primers for PCR amplification of DNA encoding an amino acid racemase as set forth in SEQ ID NO: 5 and SEQ ID NO: 6. Still further, the present invention provides a plasmid comprising DNA encoding an amino acid racemase, the DNA (i) being optimized for expression in a target microorganism with one or more codons modified in the 3′-base wobble position of the codon to be recognizable by tRNA in the target microorganism and/or (ii) having at least 80% homology with DNA set forth in SEQ ID NO: 4, excluding the 3′-base wobble position of any codon of the DNA. The present invention still further provides DNA encoding an amino acid racemase, the DNA (i) being optimized for expression in a target microorganism with one or more codons modified in the 3′-base wobble position of the codon to be recognizable by tRNA in the target microorganism and/or (ii) having at least 80% homology with DNA as set forth in SEQ ID NO: 4, excluding the 3′-base wobble position of any codon of the DNA.

The present invention further provides a process for the preparation of an (R)-β-arylalanine, the process comprising: (a) reacting in an enzyme reaction medium an (R)-α-arylalanine with an amino acid racemase to produce an (S)-α-arylalanine; and (b) reacting in the enzyme reaction medium the (S)-α-arylalanine with a phenylalanine aminomutase to produce the (R)-β-arylalanine. Still further, the amino acid racemase is encoded by DNA having (i) being optimized for expression in a target microorganism with one or more codons modified in the 3′-base wobble position of the codon to be recognizable by tRNA in the target microorganism and/or (ii) at least 80% homology with DNA as set forth in SEQ ID NO: 4, excluding the 3′-base wobble position of any codon. Further still, the phenylalanine aminomutase is encoded by DNA (i) being optimized for expression in a target microorganism with one or more codons modified in the 3′-base wobble position of the codon to be recognizable by tRNA in the target microorganism and/or (ii) having at least 80% homology with SEQ ID NO: 1, excluding the 3′-base wobble position of any codon. Still further, the amino acid racemase is encoded by DNA (i) being optimized for expression in a target microorganism with one or more codons modified in the 3′-base wobble position of the codon to be recognizable by tRNA in the target microorganism and/or (ii) having at least 80% homology with DNA as set forth in SEQ ID NO: 4, excluding the 3′-base wobble position of any codon, and wherein the phenylalanine aminomutase is encoded by DNA having (i) being optimized for expression in a target microorganism with one or more codons modified in the 3′-base wobble position of the codon to be recognizable by tRNA in the target microorganism and/or (ii) at least 80% homology with DNA as set forth in SEQ ID NO: 1, excluding the 3′-base wobble position of any codon. Further still, the amino acid racemase is produced by an E. coli deposited at MSU as MSU_racec20080826. Still further, the phenylalanine aminomutase is produced by an E. coli deposited at MSU as MSU_pamec20080804. Further still, the amino acid racemase has an amino acid sequence at least 80% homologous to SEQ ID NO: 7. Further, the phenylalanine aminomutase has an amino acid sequence at least 80% homologous to SEQ ID NO: 8. Further still, the amino acid racemase has an amino acid sequence as set forth in SEQ ID NO: 7 and wherein the phenylalanine aminomutase has an amino acid sequence as set forth in SEQ ID NO: 8. Still further, the (R)-β-arylalanine is separated from the enzyme reaction medium. Further still, the separation is chromatographic. The (R)-β-arylalanine produced in step (b) can be an essentially enantitiopure reaction product (e.g., the enantitiopure reaction product comprises at least 99 mol. % (R)-β-arylalanine relative to a combined amount of (R)-β-arylalanine and (S)-β-arylalanine produced in step (b)). In an embodiment, the reaction is conducted with step (a) before step (b). In another embodiment, step (a) and step (b) are conducted together (e.g., simultaneously with the amino acid racemase and the phenylalanine aminomutase present in the enzyme reaction mixture at the same time). The enzyme reaction medium in step (a) can initially contain (R)-α-arylalanine and be substantially free of (S)-α-arylalanine. Alternatively, the enzyme reaction medium in step (a) can initially contain (R)-α-arylalanine and (S)-α-arylalanine. Further still, the enzyme reaction medium in step (a) initially contains a racemine mixture of (R)-α-arylalanine and (S)-α-arylalanine, and the amino acid racemase maintains a 1:1 equilibrium between the (R)-α-arylalanine and the (S)-α-arylalanine enantiomers in the enzyme reaction medium as the (S)-α-arylalanine is reacted with the phenylalanine aminomutase to produce the (R)-β-arylalanine. Namely, the amino acid racemase drives the enzyme medium towards a 1:1 equilibrium even though the instantaneous distribution between (R)-α- and (S)-α-enantiomers may not be 1:1 due to the relative reaction kinetics between the racemization and resolution reactions of steps (a) and (b) (e.g., the (S)-α-enantiomer can be somewhat less than the (R)-α-enantiomer as the (S)-α-enantiomer is consumed to produce the (R)-β-arylalanine and then must be replenished in the racemization reaction).

The present invention further provides a phenylalanine aminomutase having an amino acid sequence consisting essentially of SEQ ID NO: 8. Further still, the present invention provides an amino acid racemase having an amino acid sequence at least 80% homologous to amino acid SEQ ID NO: 7.

In any of the foregoing embodiments, the relative degree of homology can be expressed alternatively or additionally as at least 85%, 90%, 93%, 95%, 97%, 98%, 99%, or 99.5% (e.g., up to 100%), independent of any microorganism-specific changes (e.g., optimization replacements at the 3′-base wobble or other positions in the codons), relative to the sequence as a whole, and/or relative to the sequence excluding the 3′-base wobble positions of sequence codons.

Use of the (R,S)-α-arylalanine mixture with the phenylalanine aminomutase converted the (S)-α-arylalanine into the corresponding (R)-β-arylalanine. During the course of the reaction, the amino acid racemase continually equilibrated the mole distribution, at 1:1, of unproductive (R)-α-arylalanine to (S)-α-arylalanine, which further supplied the phenylalanine aminomutase reaction. For instance, use of the (R,S)-α-amino-β-phenylpropionic acid mixture with the phenylalanine aminomutase converted the (S)-α-amino-β-phenylpropionic acid into the corresponding (R)-β-amino-β-phenylpropionic-amino-β-phenylpropionic acid. During the course of the reaction, the amino acid racemase continually equilibrated the mole distribution, at 1:1, of unproductive (R)-α-arylalanine with (S)-α-arylalanine, which further supplied the phenylalanine aminomutase reaction.

### BRIEF DESCRIPTION OF THE DRAWINGS

FIG. 1. Equilibration Rate of (R)-β- and (S)-α-Arylalanines by PAM Catalysis. The racemic substrates were phenylalanine (closed circle), 4′-fluorophenylalanine (open diamond), 3′-fluorophenylalanine (closed diamond), 2′-thienyl-α-alanine (closed triangle), 4′-methylphenylalanine (closed square), 2′-fluorophenylalanine (open square), 3′-methylphenylalanine (open triangle), and 2′-furanyl-α-alanine (open circle).

FIG. 2. Percent yield of biosynthetic β-arylalanines made by PAM under DKR (PAM+racemase) and KR(PAM alone) conditions for the α-arylalanine substrates phenylalanine (1), 4′-fluorophenylalanine (2), 3′-fluorophenylalanine (3), 4′-methylphenylalanine (4), 2′-thienyl-α-alanine (5), 2′-fluorophenylalanine (6), 3′-methylphenylalanine (7), and 2′-furanyl-α-alanine (8). The Figure shows the significant increase of β-arylalanine in the mixture.

FIG. 3 is a drawing of a graph showing dynamic equilibration.

FIG. 4 is a graph showing the reaction of PAM without the racemase (gray, “non-DKR”) and the reaction of PAM with the racemase (“DKR”). There is a 19% increase in the (R)-β-arylalanine.

FIG. 5 shows the aligned sequences of phenylalanine aminomutase proteins (FIG. 5A) and DNA encoding phenylalanine aminomutase proteins (FIG. 5B) used in the present invention versus those shown in the Steele reference. The optimized (OPT) DNA of SEQ ID NO: 1 expressed very well in the recombinant E. coli.

FIG. 6 are representations of chemical structures of reacting aminomutase substrates.

FIG. 7 are representations of chemical structures of compound mixtures used as starting materials.

FIG. 8 are representations of chemical structures of enantiomeric compounds produced by the coupled enzyme catalysts.

FIG. 9 Gene Sequence (SEQ ID NO: 1) for Phenylalanine Aminomutase from Taxus Canadensis. Nucleotides that represent the 3′-wobble position of all the codons are italicized and underlined.

FIG. 10 Gene Sequence (SEQ ID NO: 4) for Putative Alanine Racemase from Pseudomonas putida KT2440. Nucleotides that represent the 3′-wobble position of all of the codons are italicized and underlined.

### DESCRIPTION OF THE PREFERRED EMBODIMENTS

As used herein, the term “plasmid” means a closed-end circular DNA construct which is transferred into a microorganism cell.

The term “homologous” means at least 80%, 85%, 90%, 93%, 95%, 97%, 98%, 99%, or 99.5% to 100% homologous to DNA and protein sequences listed in SEQ ID NOS: 1 to 8 (e.g., DNA encoding an amino acid racemase or a phenylalanine aminomutase (PAM), PCR primers therefor, and proteins/enzymes produced thereby). The relative degree of homology between two sequences can be determined/expressed by including all sequence components or by excluding the 3′-base wobble position of any codon of the sequence (i.e., by excluding every third nucleotide at the 3′-base position in a codon sequence).

The term “enzymatic reaction medium” means a medium which enables the production of β-arylalanines. Although not particularly limited, the reaction medium is suitably an aqueous medium that includes one or enzymes to promote the racemization reaction (e.g., an amino acid recemase such as an arylanaline racemase) and/or the resolution reaction (e.g., a phenylalanine aminomutase) according to the disclosure. The reaction medium suitably can have a pH value ranging from neutral to basic (e.g., about 6 to 10, about 7 to 9), and the reaction medium can include a buffer system (e.g., phosphate buffer) to maintain the pH value of the reaction medium near the desired value.

The term “recombinant” means foreign DNA, which is expressed in a microorganism or plant. Preferred as a recombinant microorganism is a bacterium. Numerous genes and species can serve as a host for the plasmid of the present invention. E. coli is preferred, since its plasmids for transformation are well known.

The reference to “ATCC” is the American Type Culture Collection.

The yield of (R)-β-arylalanine in the PAM catalyzed reaction however is increased above the theoretical value by establishing dynamic kinetic resolution (DKR) conditions where the (R)-α-isomer is converted to the productive (S)-enantiomer (Scheme 1). The aryl groups at the β-position of the various alanine reactants (e.g., (R)-α-arylalanines and/or (S)-α-arylalanines) and alanine products (e.g., corresponding (R)-β-arylalanines) are not particularly limited, and generally can include any aromatic ring (e.g., 5- or 6-membered ring), substituted (e.g., one or more halo groups (such as F, Cl, Br, I), alkyl groups (such as C₁-C₁₀, C₁-C₄, or —CH₃), alkenyl groups, and/or alkynyl groups) or unsubstituted, containing no heteroatoms (i.e., a C-based ring) or containing one or more heteroatoms (e.g., O, N, S). The aryl group suitably can be represented by the generic 5- or 6-membered aromatic ring illustrated in Scheme 1 below.

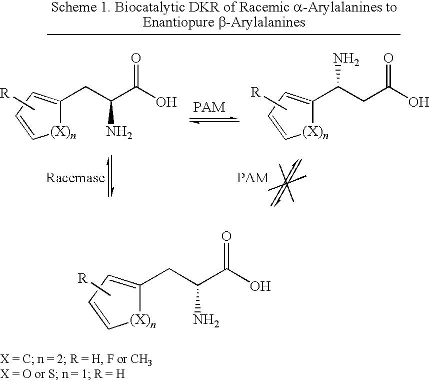

Preferably, the function of a pyridoxal-5′-phosphate-dependent amino acid racemase from Pseudomonas putida¹³ was used to maintain an (R,S)-α-arylalanine racemate in the reaction mixture. This catalyst was coupled to the resolution reaction catalyzed by PAM, and the resultant in situ DKR system enriched the availability of the productive (S)-substrate of the α-arylalanine racemate mixture, and increased the production of the (R)-β-arylalanines catalyzed by the enantioselective PAM reaction.

### EXAMPLES

### Materials and Methods

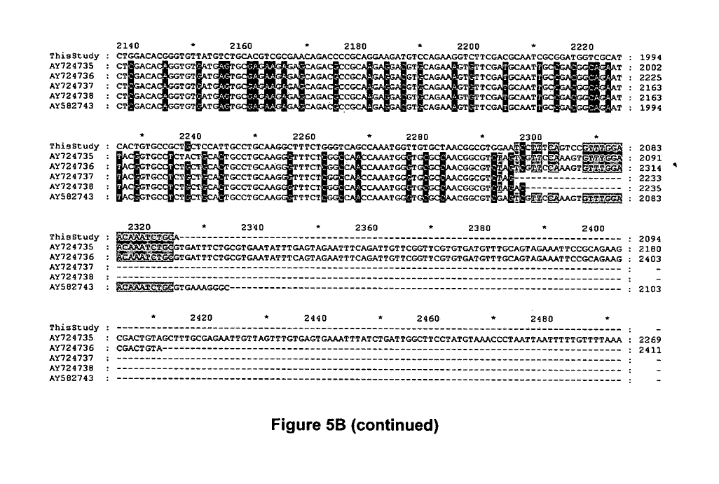

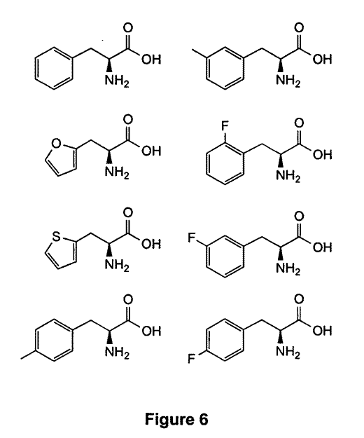

FIGS. 6, 7 and 8, show chemical structures of compounds in the Examples.

Chemicals. α-Amino acids (S)—, (R)— and (R,S)-α-phenylalanines, (S)-3-(2′-thienyl)alanine and (R,S)-3-(2′-thienyl)alanine, and (trimethylsilyl)diazomethane (2.0 M in diethyl ether) were purchased from Sigma-Aldrich-Fluka (St. Louis, Mo.). (S)-2-Amino-3-(3′-methylphenyl)propionic acid, (S)-2-amino-3-(2′-fluorophenyl)propionic acid, (S)-2-amino-3-(3′-fluorophenyl)propionic acid, (S)-2-amino-3-(4′-fluorophenyl)propionic acid, and (S)-2-amino-3-(2′-furanyl)propionic acid were obtained from Peptech (Burlington, Mass.). (S)-2-Amino-3-(4′-methylphenyl)propionic acid was purchased from Advanced ChemTech (Louisville, Ky.). The (R,S)-racemates of 2-amino-3-(2′-fluorophenyl)propionic acid, 2-amino-3-(3′-fluorophenyl)propionic acid, 2-amino-3-(4′-fluorophenyl)propionic acid were purchased from TCI (Wellesley Hills, Mass.). (R,S)-2-Amino-3-(3′-methylphenyl)propionic acid, (R,S)-2-amino-3-(4′-methylphenyl)propionic acid and (R,S)-2-amino-3-(2′-furanyl)propionic acid were synthesized by enzymatic isomerization of their corresponding (S)-amino acids; the details of the epimerization reaction are described later. β-Amino acids (R)-3-amino-3-phenylpropionic acid, (S)-3-amino-3-phenylpropionic acid, (R)-3-amino-3-(3′-methylphenyl)propionic acid, (R)-3-amino-3-(4′-methylphenyl)propionic acid, (R)-3-amino-3-(2′-fluorophenyl)propionic acid, and (R)-3-amino-3-(3′-fluorophenyl)propionic acid were obtained from Peptech (Burlington, Mass.). (R)-3-Amino-3-(4′-fluorophenyl)propionic acid was purchased from Astatech Inc. (Bristol, Pa.). The naming of the amino acids throughout the text are more generally referred to as derivatives of alanine (e.g., 2-amino-3-(4′-fluorophenyl)propionic acid is named 4′-fluorophenylalanine, and β-amino-3-(4′-methylphenyl)propionic acid is named 4′-methyl-β-phenylalanine). All other reagents were used without further purification, unless otherwise noted.

PAM Enzyme Preparation. The cDNA of the phenylalanine aminomutase, isolated from Taxus canadensis,¹⁴ was synthesized with codon optimization by DNA 2.0 (Menlo Park, Calif.) for expression in E. coli. The gene was PCR amplified with mutagenic primers that encoded NdeI (5′-CGGCATCCATATGGGTTTTGCTGTTGAATCT-3′; SEQ ID NO: 2) and BamHI (5′-CGCGGATCCTTATTATGCA-GATTTGTTCCAAAC-3′; SEQ ID NO: 3) restriction sites at the sequence termini. The resulting amplicon was cut with NdeI and BamHI and ligated in-frame into the expression vector pET28a(+) (Novagen) that was digested with the same restriction enzymes. The recombinant pET28a(+) plasmid that encoded an N-terminal His₆-tag on the PAM cDNA was verified by DNA sequencing and used to transform E. coli BL21(DE3) by standard methods.¹⁵

E. coli BL21(DE3) cells transformed to express the phenylalanine aminomutase were grown at 37° C. for 12 h in 100 mL of Luria-Bertani medium. Separate aliquots (5 mL) of this inoculum culture were then added to each of six 1-L cultures of Luria-Bertani medium supplemented with kanamycin (50 μg/mL). The cells were incubated at 37° C. until OD₆₀₀=0.7. Isopropyl-D-thiogalactopyranoside (500 μM) was added to the cultures with expression conducted at 16° C. After 16 h, the cells were harvested by centrifugation at 5,000 g (15 min), diluted in 100 mL of resuspension buffer (50 mM potassium phosphate containing 5% (v/v) glycerol and 300 mM NaCl, pH 8.0), lysed by brief sonication [five 1-min bursts at 30% power with 1 min intermittent intervals using a Branson Sonifier (Danbury, Conn.)], and the cellular debris was removed by centrifugation at 15,000 g (30 min) followed by high-speed centrifugation at 40,000 g (45 min) to remove light membrane debris. The resultant crude aminomutase in the soluble fraction was purified by Nickel-nitrilotriacetic acid (Ni-NTA) affinity chromatography according to the protocol described by the manufacturer (Qiagen, Valencia, Calif.); PAM was eluted in 250 mM imidazole. Fractions containing active soluble PAM (78 kDa) were combined and loaded into a size-selective centrifugal filtration unit (Centriprep centrifugal filter units, 30,000 MWCO; Millipore, Billerica, Mass.). The protein solution was concentrated to 1 mL and diluted several cycles until the imidazole and salt concentrations were <1 μM. The quantity of PAM and purity of the concentrated enzyme were assessed by SDS-PAGE with Coomassie Blue staining¹⁶ using Kodak 1D image analysis software (version 3.6.3) to integrate the relative intensities of the scanned protein bands with concentration standards. A Bradford assay was used to confirm the quantity of total protein.

Amino Acid Racemase Enzyme Preparation. The putative amino acid racemase (accession number AE015451 range 4,245,041-4,246,270) was selected and PCR amplified from Pseudomonas putida KT2440 (American Type Culture Collection (ATCC) (Manassas, Va.)) genomic DNA using primers that encoded NdeI (5′-AATCCATATGCCCTTTCGCCGTACCCT-3′; SEQ ID NO: 5) and BamHI (5′-CGCGGATCCTCAGTCGACGAGTATCTT-3′; SEQ ID NO: 6) restriction sites at the cDNA termini. The amplicon was digested with the appropriate restriction enzymes and sub-cloned into an identically digested expression vector pET28a(+) that encoded an N-terminal His₆-tag. Transformed E. coli BL21(DE3) cells expressing the racemase were grown, harvested, lysed, and clarified, as described above for the aminomutase, to give the soluble enzyme preparation. The crude soluble racemase was partially purified by Ni-NTA affinity chromatography, and the total protein concentration and purity were determined by the methods described earlier.

Derivatization and Quantification of Amino Acids. The amino acids in all assays described were derivatized generally as follows: to each mixture was added 0.5 N NaOH to adjust the pH to >9, and then ethyl chloroformate (200 eq, 100 μL) was added to N-acylate the arylalanines. After 10 min, the solutions were again basified (pH>9), and a second batch of ethyl chloroformate (200 eq, 100 μL) was added. After derivatization, the mixture was acidified to pH 2-3 with 6 N HCl and extracted with ethyl acetate (2×0.75 mL). The organic solvent was evaporated in vacuo, the residue was dissolved in ethyl acetate:methanol (3:1, v/v) (200 μL) [methanol was used to liberate diazomethane in the following step], and the solution was treated with excess (trimethylsilyl)diazomethane (˜5 μL) to make the methyl ester of the N-acyl amino acid.

To assess the level of the arylalanines, concentrations were calculated by coupled gas chromatography/electron-impact mass spectrometric (GC/EI-MS) analysis, and the analytes were separated on a Chirasil-D-Val column (0.25 mm inner diameter×25 m, 0.08-μm film thickness, Varian, Palo Alto, Calif.). A 1-μL aliquot of the derivatized material was loaded onto the column mounted in the GC (model 6890N, Agilent, Santa Clara, Calif.) coupled to a mass analyzer (model 5973 inert®, Agilent, Santa Clara, Calif.) in ion scan mode from 100-400 atomic mass units. The GC conditions were as follows: column temperature was held at 100° C. for 3 min, and then increased linearly at 10° C./min to 180° C. with a 3 min hold, followed by a 20° C./min linear ramp to 200° C. with a 3 min hold. Splitless injection was selected, and helium was used as the carrier gas. The relative amounts of each α-arylalanine enantiomer at equilibrium were determined by linear regression analysis of the area of the base peak ion of the derivatized α-arylalanines generated in the EI-MS. The peak area was converted to concentration by solving the corresponding linear equation, derived by plotting the area of the base peak ion (produced by the corresponding authentic standard) against concentration ranging from 0 to 1.5 mM. GC/EI-MS analysis of equimolar concentrations of derivatized (R)-α-, (S)-α-, and (R)-β-phenylalanine revealed that the abundances of the diagnostic base peak ion for each amino acid were equal.

Assessing the Racemization Rate of α-Arylalanines by the Amino Acid Racemase. The partially purified (>90%) amino acid racemase (200 μg/mL) was added to 2.5 mL of 50 mM phosphate buffer (pH 8.0) containing 5% (v/v) glycerol and one different (S)-α-arylalanine (1.5 mM) in separate assays; the mixtures were incubated at 31° C. An aliquot was withdrawn from each reaction at five time points (0.5, 10, 30, 60, and 360 min). A similar time course experiment was conducted with (R)-α-phenylalanine.

Cofactor Dependency of the Amino Acid Racemase. To assess whether sufficient PLP, made by the E. coli BL21(DE3) expression host, remained bound to the functional PLP-dependent racemase for the duration of the assay, partially purified enzyme (400 μg) was added to each of two 2-mL assays that contained (S)-α-phenylalanine (1.5 mM). To one assay, exogenous PLP (50 μM) was added, and to the other, no cofactor was added. After 6 h, the contents of the reaction vial without PLP added were divided into two fractions (0.5 mL); to one fraction was added PLP (50 μM), and the other fraction remained unchanged. Additional (S)-α-phenylalanine was added to a final concentration of 1.5 mM in all three assays, and incubated for 14 h.

Evaluation of the Effect of (R)-β-Phenylalanine on Racemase Activity. The partially purified amino acid racemase (400 μg) in 50 mM phosphate (2 mL, pH 8.0) containing 5% (v/v) glycerol buffer and (S)-α-phenylalanine (0.75 mM) was incubated in separate assays with or without (R)-β-phenylalanine (0.75 mM) added. Aliquots (0.5 mL) of each reaction mixture were withdrawn from each assay at three time points (30, 60 and 360 min). The amino acids in each fraction were derivatized and analyzed by chiral GC/EI-MS as described previously. The mol % of (S)-α-, (R)-α-arylalanine, and (R)-β-arylalanine in each sample were assessed by comparing the abundance of the diagnostic base peak fragment ion (m/z) derived by GC/EI-MS fragmentation for each of the corresponding analytes.

Assessing the Equilibration Rate of α- and β-Arylalanines by PAM. Purified PAM (250 μg) was added to 2.5 mL of 50 mM phosphate buffer (pH 8.0) containing 5% (v/v) glycerol and one different (S)-α-arylalanine (0.4 mM), or (R,S)-α-arylalanine racemate, in separate assays, and the mixtures were incubated at 31° C. An aliquot was withdrawn from each reaction at five time points (0, 3, 6, 9 and 20 h). The ratio of (R)-β-arylalanine to (S)-α-arylalanine in each aliquot was calculated by GC/EI-MS fragmentation analysis and the abundances of the base peak fragment ions of the amino acid derivatives were compared as described earlier. The relative amount of each α-arylalanine and β-arylalanine at equilibrium was determined by linear regression analysis of the area of the base peak ion of the derivatized α- and β-arylalanines generated in the EI-MS. The peak area was converted to concentration of product (or substrate) by solving the corresponding linear equation, derived by plotting the area of the base peak ion (produced by the corresponding authentic standard in the mass spectrometer) against concentration ranging from 0 to 1.5 mM.

Evaluation of the Effect of (R)-α-Phenylalanine on PAM Activity. Phenylalanine aminomutase (100 μg/mL) was added to 50 mM phosphate buffer (pH 8.0) containing 5% (v/v) glycerol at 31° C. containing either (S)-α-phenylalanine or (R)-β-phenylalanine at a range of concentrations (0.05, 0.10, 0.20, 0.40, 0.75, 1.5, and 3 mM). The reactions were incubated at 31° C. for 90 min under steady state conditions. Identical series of assays were run in parallel for the (S)-α- or (R)-β-phenylalanine as substrates, except 0.2 mM and 0.75 mM of (R)-α-phenylalanine were added separately to each series. Double reciprocal plots of velocity (vₒ) and concentration were constructed for the forward and reverse PAM reaction data sets. The equation of the best-fit line (R²=0.98) was determined (Microsoft Excel 2003, Microsoft Corporation, Redmond, Wash.) to calculate the apparent K_{M} and Kᵢ for the appropriate reaction.¹⁷

Coupled Enzyme Reaction. The coupled enzyme reaction mixture incubated at 31° C. contained partially purified amino acid racemase (200 μg) and PAM (100 μg) in 1 mL of 50 mM phosphate buffer (pH 8.0) containing 5% (v/v) glycerol. Each of the aryl α-amino acids (0.4 mM) were individually added to a separate assay tube containing the coupled enzyme mixture. After 20 h, the reactions were terminated by basification to pH 10-12 (with 0.5 N NaOH solution) and the amino acids were derivatized for quantification by chiral GC/EI-MS analysis as described previously.

Synthesis of 3-(2-Furanyl)-(R,S)-α-alanine, and 3′- and 4′-Methyl-(R,S)-α-phenylalanine. 2-Furanyl-(S)-α-alanine, 3′-methyl-(S)-α-phenylalanine and 4′-methyl-(S)-α-phenylalanine (0.5 mg, 3 μmol) were separately incubated with 400 μg of amino acid racemase (43 kDa) at 31° C. in 2 mL of 50 mM phosphate buffer (pH 8.0) containing 5% (v/v) glycerol. The reaction was incubated for 2 h, and a 0.5-mL aliquot was withdrawn from each assay, the amino acids were derivatized as described before, and the racemization was judged complete by chiral GC/EI-MS analysis. The racemic products were separated from the enzyme by size-selective centrifugal filtration (Centriprep centrifugal filter units, 10,000 MWCO; Millipore, Billerica, Mass.); the protein solution was concentrated to 10 μL and the filtrate (˜1.5 mL) was collected. The recovery of the racemic α-amino acids was quantitative as determined by linear regression analysis of the area of the base peak ion of the derivatized (S)-α-arylalanine. The isolated yield of the racemic mixtures was between 0.35 and 0.4 mg (2.2 to 2.4 μmol) at 90 to 99% based on the material remaining after 25% of the reaction volume was withdrawn earlier for chiral GC/EI-MS analysis.

### Results and Discussion

### Racemization of α-Arylalanines by Amino Acid Racemase Activity.

An amino acid racemase was cloned from the genome of Pseudomonas putida (KT2440), and was chosen for the present investigation based on its amino acid similarity to a previously reported racemase with broad specificity for naturally occurring aromatic amino acids, including but not limited to, histidine (Vᵣₑₗ=1), phenylalanine (Vᵣₑₗ=0.18), and tryptophan (Vᵣₑₗ=0.004).¹⁸ This racemase was thus anticipated to epimerize the non-natural aryl amino acids used in this investigation. The acquired racemase cDNA was heterologously expressed in E. coli BL21(DE3), and the resultant enzyme (43 kDa) was His₆-affinity purified to >90% purity and was deemed suitable for use in the assays described. Notably, based on the reported enzyme mechanism of other pseudomonad racemases,¹⁹ the pyridoxal-5′-phosphate (PLP) is preferably a cofactor of the racemase used herein; the natural reaction of this catalyst converts (S)-alanine into (R)-alanine, a key component of bacterial peptidoglycan.²⁰ The cellular concentration of unbound PLP in E. coli is estimated at ˜120 μM,²¹ and therefore, this cofactor was likely available to covalently bind and activate the amino acid racemase during recombinant expression in the bacterial host strain in this study. No change in the reaction rate of the purified racemase was observed with or without PLP supplementation in the racemization assays containing 1.5 mM α-arylalanines. This result suggested that sufficient E. coli-derived PLP remained bound in the active site of the functional racemase during the course of the assay, likely through formation of an internal Schiff-base aldamine with a conserved lysine residue (Lys-75) during protein expression.^{19,22} In addition, prior to using the racemase in a DKR context, (R)-β-phenylalanine (the product of the PAM reaction) was shown to not affect the reaction rate of the racemase during the h assay, nor was (R)-β-amino acid a substrate of the racemase.

A necessary condition to establish DKR for the biocatalytic transformation of α-arylalanine substrates to their β-isomers in the present study was to keep the substrates racemized during the resolution reaction catalyzed by the aminomutase. In individual assays, the parameters of the racemase were assessed to establish equilibrium conditions by incubating one of each α-arylalanine, known to be a substrate of the Taxus phenylalanine aminomutase.⁴ Each substituted-ring- or heterole-(S)-α-arylalanine substrate at 1.5 mM was incubated with 500 μg of the racemase in 2.5 mL assays, aliquots were withdrawn from the reactions at designated time points over 3 h, and the amino acids were derivatized as their N-ethylcarbamate methyl esters for quantitation by GC/EI-MS analysis. Both N-mono- (90-99 mol %) and N,N-dicarbamate- (1-10 mol %) derivatives were observed for each α-arylalanine substrate, and the area of the base peak fragment ion for each N-mono/N,N-dicarbamate pair were added together to account for the total mol % of each α-arylalanine enantiomer. Each α-arylalanine was found to be completely racemized by the amino acid racemase within 60 min. These data indicated that the amino acid racemase rapidly epimerized the non-natural α-arylalanines to dynamic equilibrium, and had broad substrate specificity, making this enzyme favorable for establishing DKR conditions in a one-pot reaction with PAM. In addition, the enzyme remained active for the 20 h duration of the assay as evidenced by the complete racemization of each (S)-α-arylalanine added to a racemase assay at an intermediate time point (6 h).

### Effects of (R)-β-Phenylalanine on Racemase Activity

Since the amino acid racemase would ultimately be added to a reaction in which PAM catalyzed the production of (R)-β-aminoacids, the effect of β-arylalanines on the racemase activity was studied prior to conducting the coupled enzyme assay. In general, the racemase activity on 1.5 mM of (S)-α-arylalanine was not affected by any of the β-arylalanines at 1.5 mM within the time allotted for the coupled reactions. Furthermore, (S)-β-arylalanines were not detected in any of the assays, indicating that the β-amino acids were not substrates of the racemase.

### Effect of the Non-natural (R)-α-Phenylalanine Enantiomer on PAM activity

A previous investigation demonstrated that PAM was stereospecific for (S)-α-phenylalanine, while the (R)-α-isomer was non-productive;¹² however, the inhibitory effects of the (R)-α-amino acid were not assessed.¹² Therefore, in the present investigation, to determine if the non-natural (R)-α-phenylalanine isomer affected the kinetics of PAM, the aminomutase was incubated with (S)-α-phenylalanine in the presence of 0.2 mM and 0.75 mM (R)-α-phenylalanine, and the kinetic constants were calculated. (R)-α-Phenylalanine did not significantly inhibit the PAM reaction; the apparent Michaelis constant (K_{M} apparent) for the forward reaction was 1.1 mM for the substrate tested, and the inhibition constant (Kᵢ) was 0.4 mM, without an evident change in Vₘₐₓ (0.1 μmol·min⁻¹·mg⁻¹) relative to the rate when no inhibitor was present. Comparably, in the absence of (R)-α-phenylalanine, the K_{M} of PAM was 0.7 mM, which was similar to the value previously reported.²³ In contrast, the reverse reaction was not inhibited by (R)-α-phenylalanine. Overall, the inhibition data suggests that the (R)-stereoisomer is at most a weak competitive inhibitor of PAM in the forward reaction.

### Dynamic Equilibration of α- and β-Arylalanines by the Aminomutase

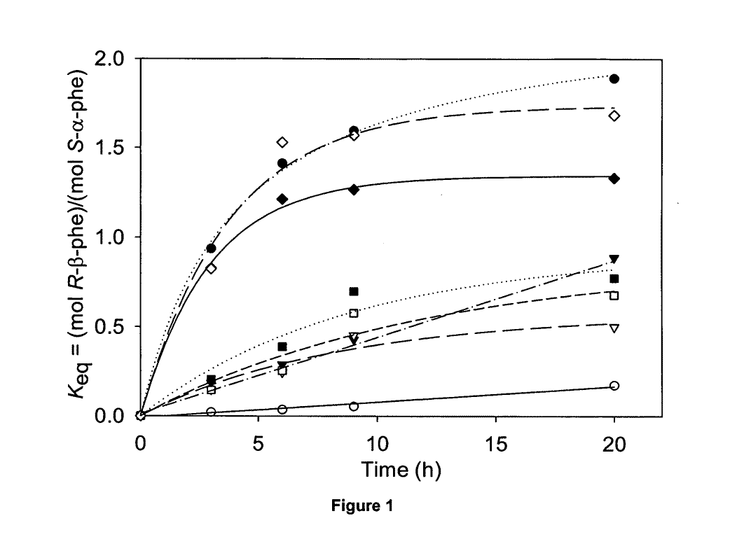

It is known that the PAM-catalyzed reaction converts (S)-α-arylalanine substrates to their respective, enantiopure (R)-β-arylalanine products (>99% e.e.).⁴ In the present study, the enantioselective PAM catalysis provided the necessary step to resolve the racemic substrate, while the racemase kept the enriched, non-productive substrate enantiomer at dynamic equilibrium with its antipode.²⁴ Parameters were established pertaining to PAM concentration and reaction time needed for each (S)-α-arylalanine of an (R,S)-racemic mixture to reach dynamic equilibrium with its β-isomeric product. Each amino acid substrate at 0.4 mM was incubated for 20 h at 31° C. with 100 μg of PAM in 1 mL of phosphate buffer. Aliquots were withdrawn from the assays at specific time intervals, and the amino acids in the reaction mixture were derivatized as their N-ethylcarbamate methyl esters for analysis by GC/EI-MS. Under these conditions, all of the (R)-β-arylalanine products approached maximum accumulation except for the heteroaromatic amino acid substrates, 2′-furanyl-α-alanine and 2′-thienyl-α-alanine; K_{eq}=1.8 was calculated for PAM with its natural substrate (FIG. 1).

The ratio of the heterole-β-arylalanines to their counterpart heterole-α-arylalanines ((S)-2-thienyl-α-alanine or (S)-2-furanyl-α-alanine) established by PAM, remained at steady state over 20 h. This indicated that the reactions containing these amino acids were slower and therefore had not yet reached equilibrium. The β-product to α-substrate ratio for these heterole arylalanines was at 0.71 and 0.41, respectively, after 20 h, and reflected that the steady-state turnover rate (vₒ) at the stop-point for the conversion of (S)-2-thienyl-α-alanine and (S)-2-furanyl-α-alanine to their respective β-isomers by PAM was greater for the former compared to the latter (FIG. 1). The racemic substrates were phenylalanine, 4′-fluorophenylalanine, 3′-fluorophenylalanine, 2′-thienyl-α-alanine, 4′-methylphenylalanine, 2′-fluorophenylalanine, 3′-methylphenylalanine, and 2′-furanyl-α-alanine. This finding was consistent with kinetics data reported in a previous study for these heterole aromatic amino acids.⁴ Presently, the basis for this difference in turnover rate of the heterole-α-arylalanines by PAM remains unknown. Furthermore, the variability of the K_{eq} values for various substrates is intriguing; however, it is presently unclear whether inductive or steric effects are influencing the position of the equilibrium for the various substituted-arylalanine regioisomers.

Curiously, the K_{eq} of PAM calculated in the current study is at 1.8, and is ˜1.6-fold greater than that calculated (K_{eq}=1.1) for PAM at the same temperature in a previous study with enantiopure (S)-α-phenylalanine as the substrate.⁴ This increase might reflect the higher enzyme (100 μg/mL) and substrate (400 μM) concentrations used in each assay, in this study, which represented an approximate order of magnitude increase in concentration of both parameters compared to the 10 μg/mL enzyme and 10 μM substrate used in the previous investigation.¹² The elevated concentrations used herein increased the yield of the biosynthetic product, and therefore improved the signal-to-noise of the derivatized amino acids isolated from the assay mixtures and analyzed by GC/EI-MS.

### Production of enantiopure β-arylalanines in coupled racemase/aminomutase assays

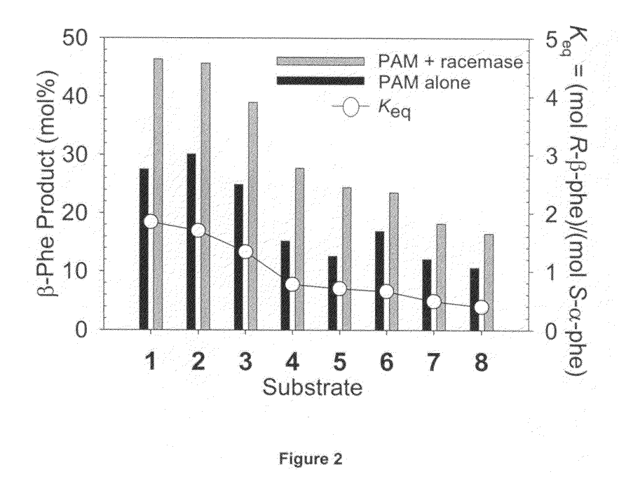

The enzyme reaction conditions described above, established equilibrium parameters needed to increase the production of (R)-β-arylalanines by a DKR process. Assays were conducted to directly compare the conversion of α-arylalanines to β-arylalanines in the presence and absence of racemase. In the first set of assays, each (R,S)-α-arylalanine (0.4 mM) racemate was separately incubated with 100 μg of PAM in 2 mL of phosphate buffer for 20 h to establish dynamic equilibrium. Synthetic derivatization of the amino acids to their carbamate methyl esters, followed by GC/EI-MS analysis of these products showed that between 11 and 30 mol % of the various (R)-β-arylalanines were produced relative to the α-arylalanine racemate (FIG. 2). The racemic-α-arylalanine substrates are phenylalanine, 4′-fluorophenylalanine, 3′-fluorophenylalanine, 4′-methylphenylalanine, 2-thienyl-α-alanine, 2′-fluorophenylalanine, 3′-methylphenylalanine, and 2-furanyl-α-alanine. The relative mol % of the (S)-α-arylalanines was consistent with the equilibrium constants for PAM and the respective (S)-α/(R)-β-arylalanine pairs established earlier, in this study; the mol % of the (R)-α-arylalanines in each assay remained virtually unchanged, as these enantiomers were not isomerized by PAM.

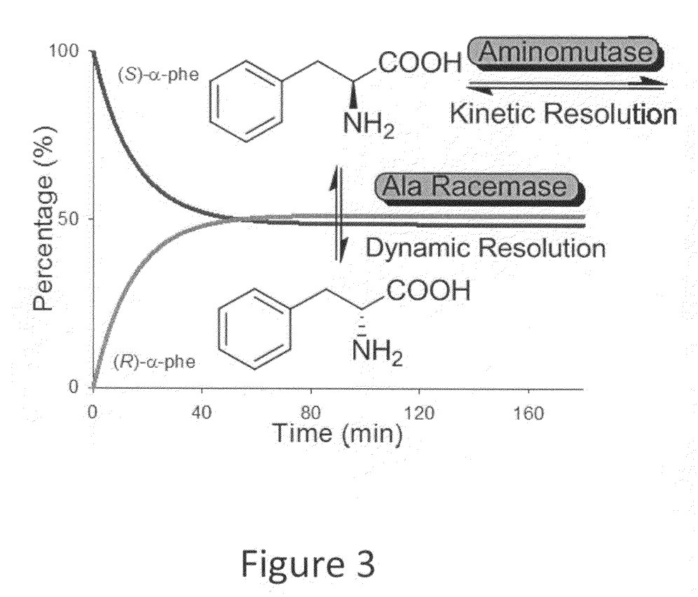

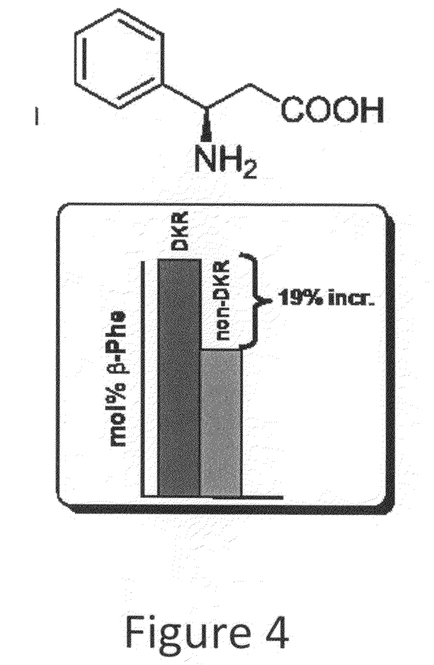

In a separate complementary set of 2 mL assays, 0.4 mM of each racemic α-arylalanine was separately mixed with 200 μg/mL of the amino acid racemase and 100 μg/mL of PAM to establish DKR conditions. After incubating for 20 h, the amino acids were derivatized for GC/EI-MS analysis, as described previously. Examination of the analytes showed that the various (R)-β-arylalanines were between 17 to 49 mol % relative to the mol % of the (R,S)-α-arylalanine racemates, when the amino acid racemase and aminomutase activities were coupled in the same reaction. This represented a 6 to 19% increase in conversion (depending on the substrate) of the (R,S)-α-arylalanines to (R)-β-arylalanines under the DKR reaction conditions. The natural α-phenylalanine substrate showed the largest increase (19%; FIGS. 3 and 4) in conversion to β-phenylalanine under DKR conditions (followed by the 4′-fluoro- (16%), 3′-fluoro- (14%), 4′-methyl- (12%), and the 2′-thienyl- (12%) α-arylalanines) compared to the conversion of the α-arylalanines under kinetic resolution conditions with PAM alone (FIG. 2). Most likely, the higher isomerization rate of the 3′- and 4′-fluoro substrates by PAM catalysis, compared to the α-amino acids isomerized more slowly,⁴ contributes to the relatively higher production of the corresponding fluoro-β-amino acids under DKR conditions.

In reference to the DNA sequences, SEQ ID NO: 9 is the wild type phenylalanine aminomutase gene deposited in Genbank (Accession No. 582743).¹⁴ SEQ ID NO: 1 is the codon optimized phenylalanine aminomutase DNA cloned into a plasmid that was utilized to generate the phenylalanine aminomutase protein used in the enzyme catalysis.

The 3′-nucleotides of the codons of the DNA that correspond to the 5′-base (i.e., the “wobble position”) of the respective anitcodon of the tRNAs are excluded from the sequence evaluation when comparing SEQ ID NOS: 1 and 4 to their wild-type DNA sequences. A tRNA delivers a particular amino acid to the polypeptide (enzyme) chain encoded by the DNA strand. Since a tRNA that delivers a particular amino acid can pair with more than one codon, then conceivably DNA sequences can be highly variable (at least 80% homologous) and still encode the same polypeptide chain (e.g., codons with the same first two nucleotides, but different third position nucleotides in the wobble position of the codon, can code for the same amino acids). The sequence of the PAM DNA was altered from its original sequence (SEQ ID NO: 9) to SEQ ID NO: 1 for optimal expression of the PAM gene in E. coli. This optimization was achieved by exchanging the 3-base “wobble position” of the PAM DNA codons to codon that were recognizable by the tRNA found in E. coli. The amino acid racemase DNA (SEQ ID NO: 4) can be optimized for expression in E. coli.

The frequencies with which different codons appear in E. coli genes are different than genes derived from plants, for example. The concentration of a specific tRNA is proportional to the frequency of the codon usage in the all genes present in the E. coli genome. A rarely used codon will ordinarily be present in a foreign gene, derived from the same or different genus, transferred into E. coli for expression, and the E. coli will likely have a relatively lower concentration of the requisite tRNA to recognize the codon. Therefore, genes that contain codons rare in E. coli may be inefficiently expressed. Rare codons can prematurely terminate protein synthesis or incorporate incorrect amino acids. In addition, rare codons positioned in close proximity increase the frequency of translation errors and can further reduce the expression level.

SEQ ID NOS: 10, 11, 12 and 13 are the phenylalanine aminomutase DNAs outlined in the patent disclosed by Steele.³⁰ The genes are designated based on the name of the depositor (in parentheses), the plant source of the gene (italics), and the NCBI databank accession number which are incorporated herein in their entireties.

In reference to the amino acid sequences used in the Steele patent (SEQ ID NO: 14) there is a 98% similarity to SEQ ID NO:8 (present invention).

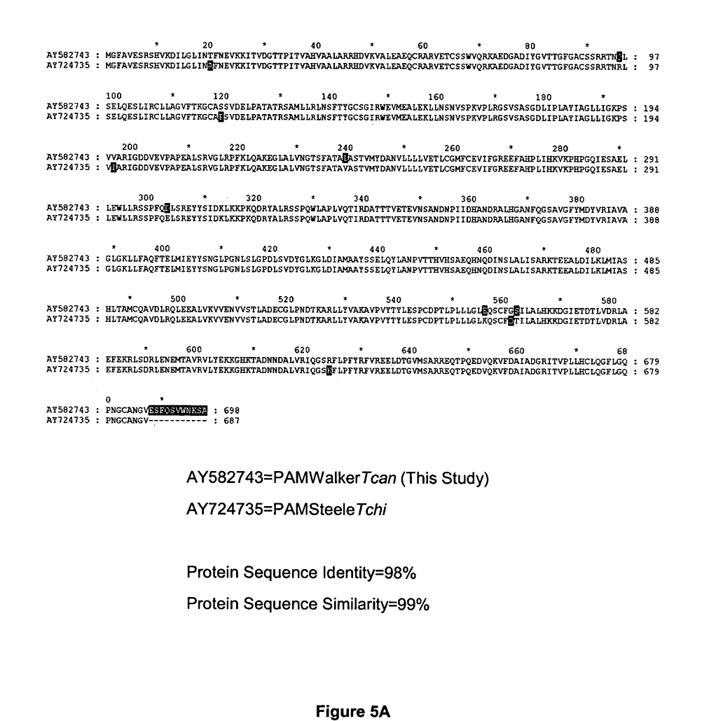

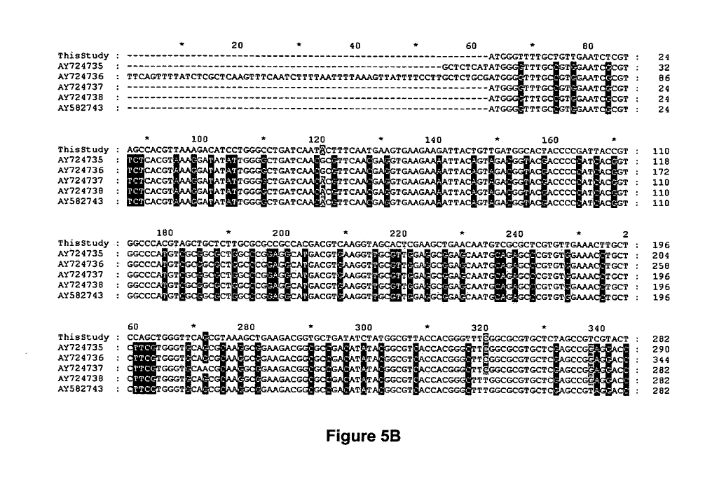

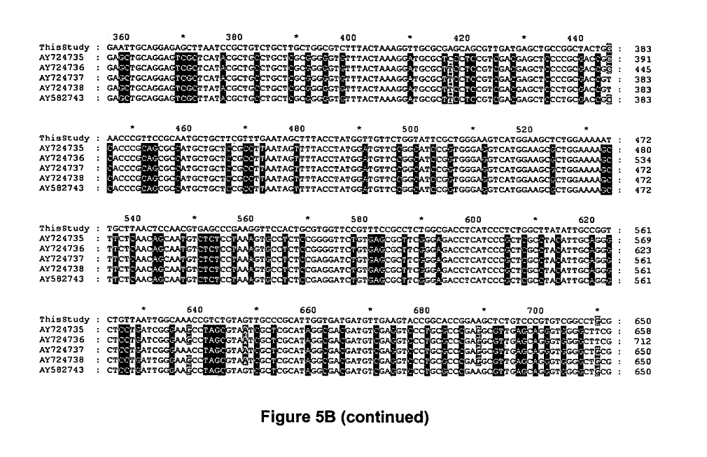

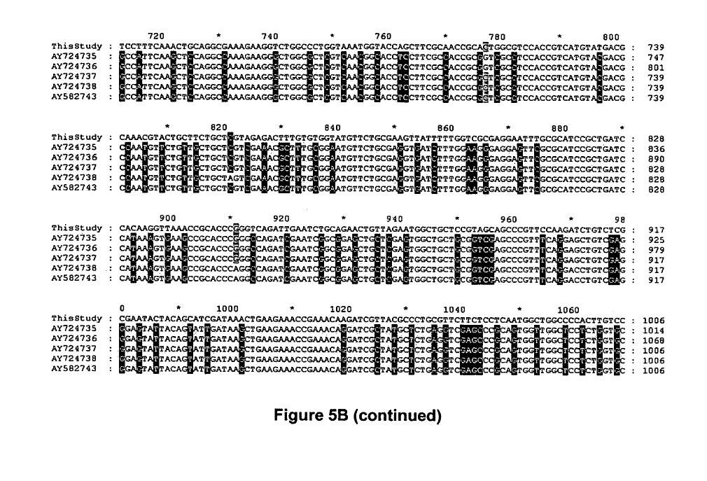

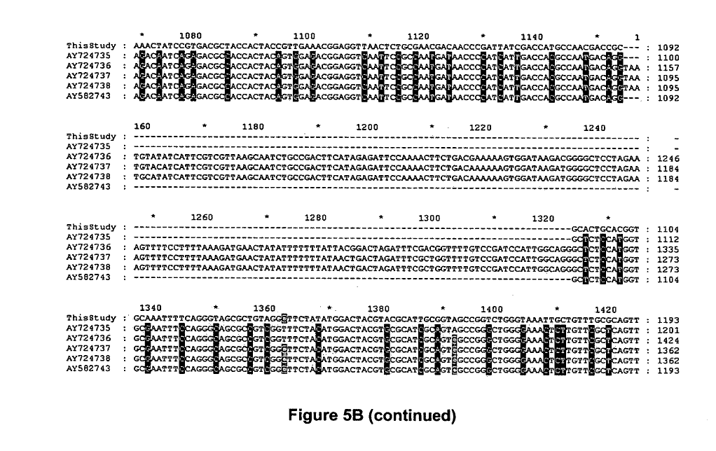

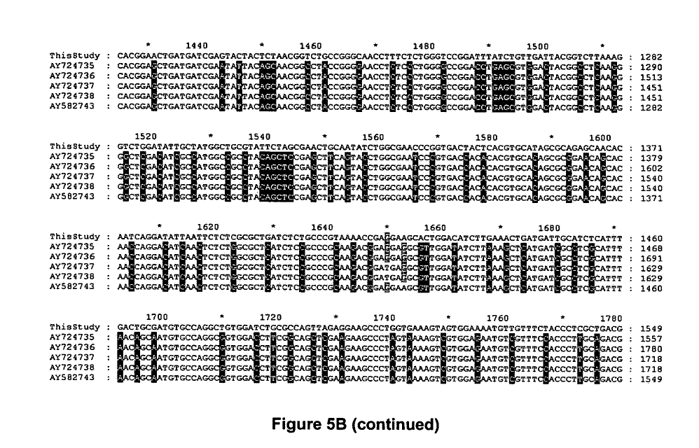

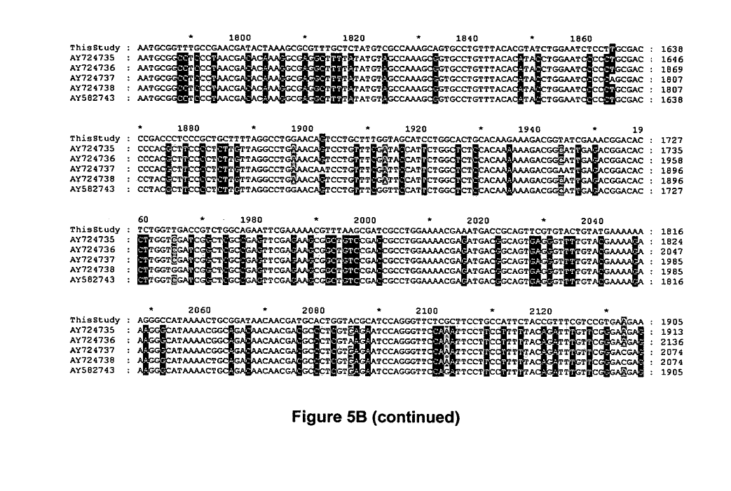

In FIG. 5 the sequence alignments and % identity and % similarity for the protein sequences and % identity for the DNA sequences are set forth. The naming is outlined in a key on the alignments that correspond to the NCBI databank accession number. The percentage Gene Sequence Identity is set forth in Table 1.

TABLE 1

| Present Disclosure Sequence | Reference Sequence | Relative Sequence Identity |
|---|---|---|
| This Study = PAMWalkerTcan OPT | AY724735 = PAMSteeleTchi | Gene Sequence Identity = 74% |
| This Study = PAMWalkerTcan OPT | AY724736 = PAMSteeleTchi2 | Gene Sequence Identity = 76% |
| This Study = PAMWalkerTcan OPT | AY724737 = PAMSteeleTx | Gene Sequence Identity = 75% |
| This Study = PAMWalkerTcan OPT | AY724738 = PAMSteeleTcan | Gene Sequence Identity = 75% |
| This Study = PAMWalkerTcan OPT | AY582743 = PAMWalkerTcan | Gene Sequence Identity = 75% |
| AY582743 = PAMWalkerTcan | AY724735 = PAMSteeleTchi | Protein Sequence Identity = 98% |

“OPT” means optimal

The protein Sequence identity for SEQ ID NOS: 1 and 14 is 98% and the Sequence Similarity is 99%.

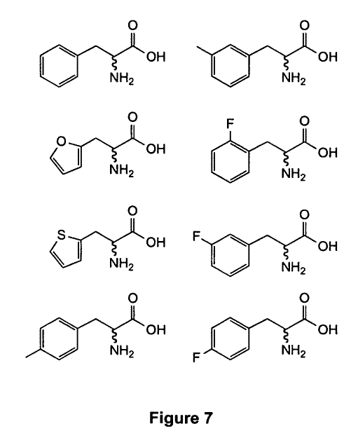

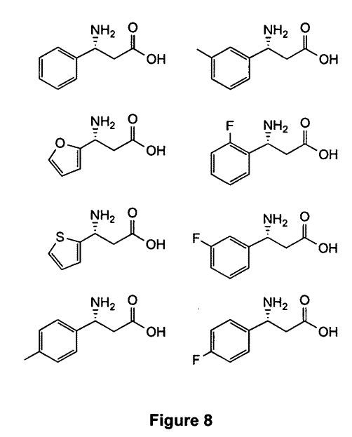

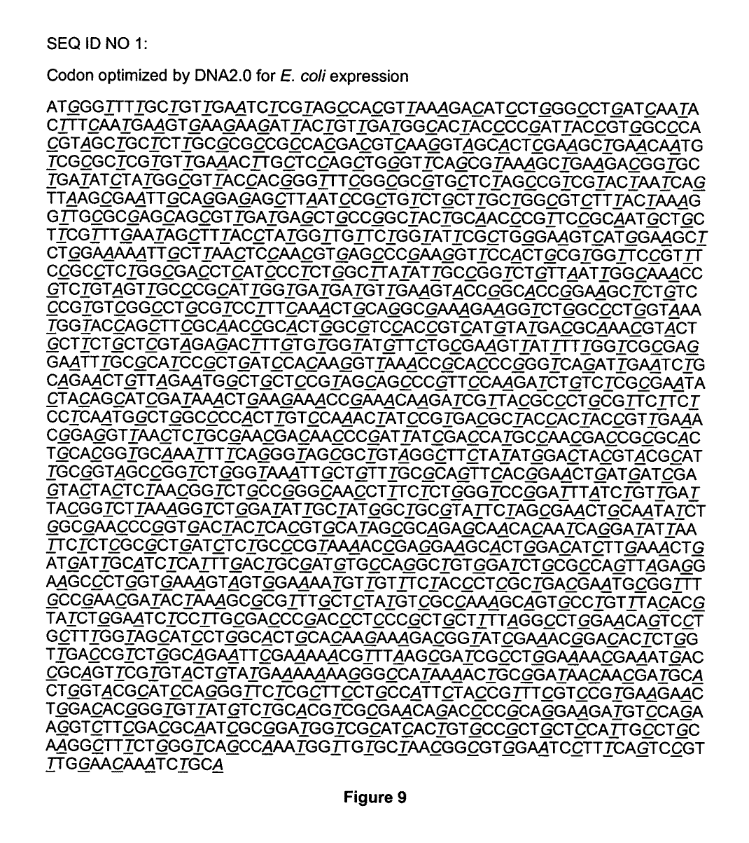

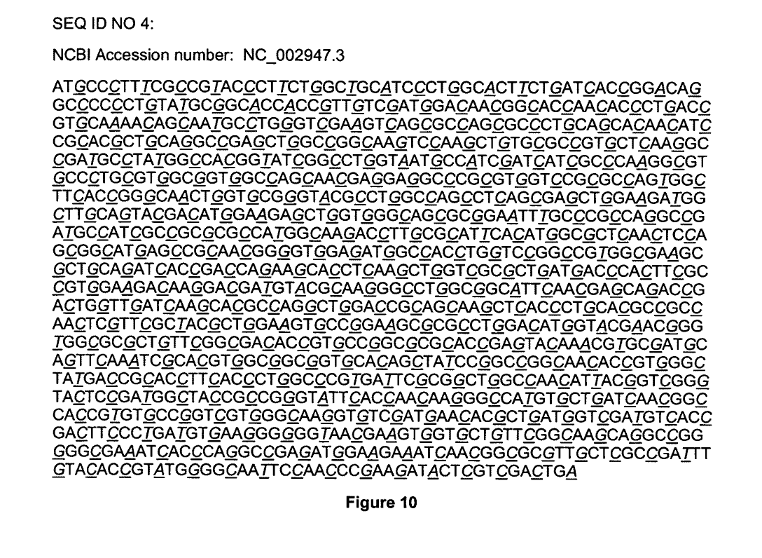

FIG. 6 are the substrates of the phenylalanine aminomutase that are enantiomerically pure, having (S)-stereochemistry at the α-carbon position. The picture of the actual mixture of two enantiomeric substrates utilized in the coupled enzyme assay is shown in representation FIG. 7 where the (R,S)-mixture of the amino acid substrates is indicated by placing a wavy line between the nitrogen and α-carbon. In representation FIG. 8 are the enantiomerically pure products made in the coupled enzyme catalysis that are now β-amino acids with (R)-stereochemistry at the β-carbon.

### CONCLUSION

The Taxus phenylalanine aminomutase (PAM) enzyme converts several (S)-α-arylalanines to their corresponding (R)-β-arylalanines. After incubating various chiral or racemic substrates with 100 μg of PAM for 20 h at 31° C., each (S)-α-arylalanine was enantioselectively isomerized to its corresponding (R)-β-product. With racemic starting materials, the ratio of (R)-β-arylalanine product to the (S)-α-substrate ranged between 0.4 and 1.8, and the remaining non-productive (R)-α-arylalanine became enriched. To utilize the (R)-α-isomer, the catalysis of a promiscuous amino acid racemase from Pseudomonas putida (KT2440) was coupled with that of PAM to establish dynamic kinetic resolution (DKR) conditions. The inclusion of a biocatalytic racemization along with the PAM-catalyzed reaction significantly increased the yield of the enantiopure β-arylalanines from racemic α-arylalanine substrates between 6 and 19%, depending on the arylalanine. This combination of catalysts potentially has important application in the production of chiral β-arylalanine building blocks.

Coupling a broad-spectrum amino acid racemase with a promiscuous, yet enantioselective aminomutase in the dynamic kinetic resolution reactions, described herein, was key towards significantly increasing the conversion yields of (R)-β-arylalanines (at >99% e.e.) from racemic α-arylalanines. Semi-biochemical dynamic kinetic resolution is finding a niche as a technique for asymmetric synthesis that includes innovative methods for enzymatic catalysis that optimize the yields of enantiomerically-enriched products.²⁵⁻²⁹ The capacity to obtain one enantiomer (or diastereomer) from a racemic mixture is attractive especially if the equilibrium can be further shifted towards the product, and the DKR process that was developed has significant potential in the production of β-amino acid scaffolds to construct bioactive chiral molecules.

The coupled enzyme reaction has no supplemented cofactors in the aqueous medium. The phosphate is added to buffer the medium against pH changes, and it does not serve as a cofactor or cosubstrate for the enzymatic reaction process described herein. Many other compounds can be used as a buffer if they do not affect the function of the enzymes. Glycerol is also not essential for operation of the reaction mixture; this additive likely contributes to prolonging the stability of the structure/function of the enzyme. The aminomutase reaction has been observed to work within a pH from neutral to basic; the racemase is also functional within this pH range. Thus, the medium composition can be variable and the function of both the aminomutase and racemase are maintained under identical reaction mixture conditions.

It is intended that the foregoing description be only illustrative of the present invention and that the present invention be limited only by the hereinafter appended claims.

### REFERENCES INCORPORATED BY REFERENCE IN THEIR ENTIRETIES

(1) Spiteller, P.; Von Nussbaum, F. In Enantioselective Synthesis of b-Amino Acids Juaristi, E., Soloshonok, V. A., Eds.; John Wiley & Sons, Inc.: Hoboken, N.J., 2005, p 19-91.
(2) Weaver, D. F.; Tan, C. Y. K.; Kim, S. T.; Kong, X.; Wei, L.; Carran, J. R. WO 2002073208, Queen's University at Kingston, Can.; Neurochem Inc., 2002, 122 pp.
(3) Mohr, J. T.; Krout, M. R.; Stoltz, B. M. Nature 2008, 455, 323-332.
(4) Klettke, K. L.; Sanyal, S.; Mutatu, W.; Walker, K. D. J. Am. Chem. Soc. 2007, 129, 6988-6989.
(5) Parry, T. J.; Brosius, R.; Thyagarajan, R.; Carter, D.; Argentieri, D.; Falotico, R.; Siekierka, J. Eur. J. Pharmacol. 2005, 524, 19-29.
(6) Fischer, F. J.; Miller, J. W.; Andrews, M. O. US 2006030826, CIP of U.S. Pat. No. 410,587, Cook Urological Incorporated, 2006, 12 pp.
(7) Joergensen, E.; Kelbaek, H. Eur. Heart J. 2005, 26, 1049-1051.
(8) Zakharian, T. Y.; Seryshev, A.; Sitharaman, B.; Gilbert, B. E.; Knight, V.; Wilson, L. J. J. Am. Chem. Soc. 2005, 127, 12508-12509.
(9) Johannsen, M.; Sachs, M.; Roigas, J.; Hinke, A.; Staack, A.; Loening, S. A.; Schnorr, D.; Wille, A. H. Eur. Urol. 2005, 48, 246-251.
(10) Rouzier, R.; Perou, C. M.; Symmans, W. F.; Ibrahim, N.; Cristofanilli, M.; Anderson, K.; Hess, K. R.; Stec, J.; Ayers, M.; Wagner, P.; Morandi, P.; Fan, C.; Rabiul, I.; Ross, J. S.; Hortobagyi, G. N.; Pusztai, L. Clin. Cancer. Res. 2005, 11, 5678-5685.
(11) Mutatu, W.; Klettke, K. L.; Foster, C.; Walker, K. D. Biochemistry 2007, 46, 9785-9794.
(12) Walker, K. D.; Klettke, K.; Akiyama, T.; Croteau, R. J. Biol. Chem. 2004, 279, 53947-53954.
(13) Bechtold, M.; Makart, S.; Reiss, R.; Alder, P.; Panke, S. Biotechnol. Bioeng. 2007, 98, 812-824.
(14) Jennewein, S.; Wildung, M. R.; Chau, M.; Walker, K.; Croteau, R. Proc. Natl. Acad. Sci. U.S.A. 2004, 101, 9149-9154.
(15) Ausubel, F. M.; Brent, R.; Kingston, R. E.; Moore, D. D.; Seidman, G.; Smith, J. A.; Struhl, K. Short Protocols in Molecular Biology, 5th Edition, 2002.
(16) Wray, W.; Boulikas, T.; Wray, V. P.; Hancock, R. Anal. Biochem. 1981, 118, 197-203.
(17) Fersht, A. Enzyme structure and mechanism; 2nd ed.; W.H. Freeman: New York, 1985.
(18) Kino, K.; Sato, M.; Yoneyama, M.; Kirimura, K. Appl. Microbiol. Biotechnol. 2007, 73, 1299-1305.
(19) Schnell, B.; Faber, K.; Kroutil, W. Adv. Synth. Catal. 2003, 345, 653-666.
(20) Walsh, C. J. Biol. Chem. 1989, 264, 2393-2396.
(21) Fu, T.-F.; di Salvo, M.; Schirch, V. Anal. Biochem. 2001, 298, 314-321.
(22) Shaw, J. P.; Petsko, G. A.; Ringe, D. Biochemistry 1997, 36, 1329-1342.
(23) Steele, C. L.; Chen, Y.; Dougherty, B. A.; Li, W.; Hofstead, S.; Lam, K. S.; Xing, Z.; Chiang, S.-J. Arch. Biochem. Biophys. 2005, 438, 1-10.
(24) Adams, E.; Mukherjee, K.; Dunathan, H. Arch. Biochem. Biophys. 1974, 165, 126-132.
(25) Yamaguchi, S.; Komeda, H.; Asano, Y. Appl. Environ. Microbiol. 2007, 73, 5370-5373.
(26) Borén, L.; Martin-Matute, B.; Xu, Y.; Cordova, A.; Backvall, J.-E. Chem. Eur. J. 2006, 12, 225-232.
(27) May, 0.; Verseck, S.; Bommarius, A.; Drauz, K. Org. Process Res. Dev. 2002, 6, 452-457.
(28) Huerta, F. F.; Minidis, A. B. E.; Backvall, J. E. Chem. Soc. Rev. 2001, 30, 321-331.
(29) Gotor, V.; Alfonso, I.; García-Urdiales, E. Asymmetric Organic Synthesis with Enzymes; Wiley-VCH: Weinheim, 2008.
(30) Steele et al WO 03/066871A2, Published Aug. 14, 2003.

We claim:

1. E. coli cells comprising a transformed plasmid encoding phenylalanine aminomutase deposited at MSU as MSU_pamec20080804.

2. A recombinant microorganism comprising a transformed plasmid encoding phenylalanine aminomutase, the plasmid comprising DNA (i) being optimized for expression in the microorganism with one or more codons modified in the 3′-base wobble position of the codon to be recognizable by tRNA in the microorganism and (ii) having at least 80% homology with DNA as set forth in SEQ ID NO: 1, excluding the 3′-base wobble position of any codon of the DNA.

3. DNA primers for PCR amplification of DNA for encoding phenylalanine aminomutase as set forth in SEQ ID NO: 2 and SEQ ID NO: 3.

4. A plasmid comprising DNA encoding phenylalanine aminomutase, the DNA (i) being optimized for expression in a target microorganism with one or more codons modified in the 3′-base wobble position of the codon to be recognizable by tRNA in the target microorganism and (ii) having at least 80% homology with DNA as set forth in SEQ ID NO: 1, excluding the 3′-base wobble position of any codon of the DNA.

5. DNA encoding phenylalanine aminomutase, the DNA (i) being optimized for expression in a target microorganism with one or more codons modified in the 3′-base wobble position of the codon to be recognizable by tRNA in the target microorganism and (ii) having at least 80% homology with DNA as set forth in SEQ ID NO: 1, excluding the 3′-base wobble position of any codon of the DNA.

6. E. coli cells comprising a transformed plasmid encoding an amino acid racemase deposited at MSU as MSU_racec20080826.

7. A recombinant microorganism comprising a transformed plasmid encoding an amino acid racemase, the plasmid comprising DNA (i) being optimized for expression in the microorganism with one or more codons modified in the 3′-base wobble position of the codon to be recognizable by tRNA in the microorganism and (ii) having at least 80% homology with DNA set forth in SEQ ID NO: 4, excluding the 3′-base wobble position of any codon of the DNA.

8. DNA primers for PCR amplification of DNA encoding an amino acid racemase as set forth in SEQ ID NO: 5 and SEQ ID NO: 6.

9. A plasmid comprising DNA encoding an amino acid racemase, the DNA (i) being optimized for expression in a target microorganism with one or more codons modified in the 3′-base wobble position of the codon to be recognizable by tRNA in the target microorganism and (ii) having at least 80% homology with DNA set forth in SEQ ID NO: 4, excluding the 3′-base wobble position of any codon of the DNA.

10. DNA encoding an amino acid racemase, the DNA (i) being optimized for expression in a target microorganism with one or more codons modified in the 3′-base wobble position of the codon to be recognizable by tRNA in the target microorganism and (ii) having at least 80% homology with DNA as set forth in SEQ ID NO: 4, excluding the 3′-base wobble position of any codon of the DNA.

11. A process for the preparation of an (R)-β-arylalanine, the process comprising:
(a) reacting in an enzyme reaction medium an (R)-α-arylalanine with an amino acid racemase to produce an (S)-α-arylalanine; and
(b) reacting in the enzyme reaction medium the (S)-α-arylalanine with a phenylalanine aminomutase to produce the (R)-β-arylalanine.

12. The process of claim 11 wherein the amino acid racemase is encoded by DNA (i) being optimized for expression in a target microorganism with one or more codons modified in the 3′-base wobble position of the codon to be recognizable by tRNA in the target microorganism and (ii) having at least 80% homology with DNA as set forth in SEQ ID NO: 4, excluding the 3′-base wobble position of any codon of the DNA.

13. The process of claim 11 wherein the phenylalanine aminomutase is encoded by DNA (i) being optimized for expression in a target microorganism with one or more codons modified in the 3′-base wobble position of the codon to be recognizable by tRNA in the target microorganism and (ii) having at least 80% homology with SEQ ID NO: 1, excluding the 3′-base wobble position of any codon of the DNA.

14. The process of claim 11 wherein the amino acid racemase is encoded by DNA (i) being optimized for expression in a target microorganism with one or more codons modified in the 3′-base wobble position of the codon to be recognizable by tRNA in the target microorganism and (ii) having at least 80% homology with DNA as set forth in SEQ ID NO: 4, excluding a 3′-base wobble position of any codon of the DNA, and wherein the phenylalanine aminomutase is encoded by DNA (i) being optimized for expression in a target microorganism with one or more codons modified in the 3′-base wobble position of the codon to be recognizable by tRNA in the target microorganism and (ii) having at least 80% homology with DNA as set forth in SEQ ID NO: 1, excluding a 3′-base wobble position of any codon of the DNA.

15. The process of claim 11 wherein the amino acid racemase is produced by an E. coli deposited at MSU as MSU_racec20080826.

16. The process of claim 11 wherein the phenylalanine aminomutase is produced by an E. coli deposited at MSU as MSU_pamec20080804.

17. The process of claim 11 wherein the amino acid racemase has an amino acid sequence at least 80% homologous to SEQ ID NO: 7.

18. The process of claim 11 wherein the phenylalanine aminomutase has an amino acid sequence at least 80% homologous to SEQ ID NO: 8.

19. The process of claim 11 wherein the amino acid racemase has an amino acid sequence as set forth in SEQ ID NO: 7 and wherein the phenylalanine aminomutase has an amino acid sequence as set forth in SEQ ID NO: 8.

20. The process of claim 11, further comprising:
(c) separating the (R)-β-arylalanine from the enzyme reaction medium.

21. The process of claim 20 wherein the separation is chromatographic.

22. The process of claim 11 wherein the (R)-β-arylalanine produced in step (b) is an essentially enantiopure reaction product.

23. The process of claim 22 wherein the enantiopure reaction product comprises at least 99 mol. % (R)-β-arylalanine relative to a combined amount of (R)-β-arylalanine and (S)-β-arylalanine produced in step (b).

24. The process of claim 11 comprising performing step (a) before step (b).

25. The process of claim 11 comprising performing step (a) and step (b) together.

26. The process of claim 11 wherein the enzyme reaction medium in step (a) initially contains (R)-α-arylalanine and is substantially free of (S)-α-arylalanine.

27. The process of claim 11 wherein the enzyme reaction medium in step (a) initially contains (R)-α-arylalanine and (S)-α-arylalanine.

28. The process of claim 11 wherein the enzyme reaction medium in step (a) initially contains a racemine mixture of (R)-α-arylalanine and (S)-α-arylalanine, and the amino acid racemase maintains a 1:1 equilibrium between the (R)-α-arylalanine and the (S)-α-arylalanine enantiomers in the enzyme reaction medium as the (S)-α-arylalanine is reacted with the phenylalanine aminomutase to produce the (R)-β-arylalanine.

29. A phenylalanine aminomutase having an amino acid sequence consisting essentially of SEQ ID NO: 8.

30. An amino acid racemase having an amino acid sequence at least 80% homologous to amino acid SEQ ID NO: 7.


SEQUENCE LISTING (14 sequences)

SEQ ID NO: 1 - DNA, 2094 bp, Taxus canadensis
  misc_feature: Gene Sequence for Phenylalanine Aminomutase from Taxus canadensis  Codon optimized by DNA2.0 for E. coli expression
atgggttttgctgttgaatctcgtagccacgttaaagacatcctgggcctgatcaatactttcaatgaagtgaagaagattactgttgatggcactaccccgattaccgtggcccacgtagctgctcttgcgcgccgccacgacgtcaaggtagcactcgaagctgaacaatgtcgcgctcgtgttgaaacttgctccagctgggttcagcgtaaagctgaagacggtgctgatatctatggcgttaccacgggtttcggcgcgtgctctagccgtcgtactaatcagttaagcgaattgcaggagagcttaatccgctgtctgcttgctggcgtctttactaaaggttgcgcgagcagcgttgatgagctgccggctactgcaacccgttccgcaatgctgcttcgtttgaatagctttacctatggttgttctggtattcgctgggaagtcatggaagctctggaaaaattgcttaactccaacgtgagcccgaaggttccactgcgtggttccgtttccgcctctggcgacctcatccctctggcttatattgccggtctgttaattggcaaaccgtctgtagttgcccgcattggtgatgatgttgaagtaccggcaccggaagctctgtcccgtgtcggcctgcgtcctttcaaactgcaggcgaaagaaggtctggccctggtaaatggtaccagcttcgcaaccgcactggcgtccaccgtcatgtatgacgcaaacgtactgcttctgctcgtagagactttgtgtggtatgttctgcgaagttatttttggtcgcgaggaatttgcgcatccgctgatccacaaggttaaaccgcacccgggtcagattgaatctgcagaactgttagaatggctgctccgtagcagcccgttccaagatctgtctcgcgaatactacagcatcgataaactgaagaaaccgaaacaagatcgttacgccctgcgttcttctcctcaatggctggccccacttgtccaaactatccgtgacgctaccactaccgttgaaacggaggttaactctgcgaacgacaacccgattatcgaccatgccaacgaccgcgcactgcacggtgcaaattttcagggtagcgctgtaggcttctatatggactacgtacgcattgcggtagccggtctgggtaaattgctgtttgcgcagttcacggaactgatgatcgagtactactctaacggtctgccgggcaacctttctctgggtccggatttatctgttgattacggtcttaaaggtctggatattgctatggctgcgtattctagcgaactgcaatatctggcgaacccggtgactactcacgtgcatagcgcagagcaacacaatcaggatattaattctctcgcgctgatctctgcccgtaaaaccgaggaagcactggacatcttgaaactgatgattgcatctcatttgactgcgatgtgccaggctgtggatctgcgccagttagaggaagccctggtgaaagtagtggaaaatgttgtttctaccctcgctgacgaatgcggtttgccgaacgatactaaagcgcgtttgctctatgtcgccaaagcagtgcctgtttacacgtatctggaatctccttgcgacccgaccctcccgctgcttttaggcctggaacagtcctgctttggtagcatcctggcactgcacaagaaagacggtatcgaaacggacactctggttgaccgtctggcagaattcgaaaaacgtttaagcgatcgcctggaaaacgaaatgaccgcagttcgtgtactgtatgaaaaaaagggccataaaactgcggataacaacgatgcactggtacgcatccagggttctcgcttcctgccattctaccgtttcgtccgtgaagaactggacacgggtgttatgtctgcacgtcgcgaacagaccccgcaggaagatgtccagaaggtcttcgacgcaatcgcggatggtcgcatcactgtgccgctgctccattgcctgcaaggctttctgggtcagccaaatggttgtgctaacggcgtggaatcctttcagtccgtttggaacaaatctgca

SEQ ID NO: 2 - DNA, 31 bp, Artificial Sequence
  Synthetic primer
cggcatccatatgggttttgctgttgaatct

SEQ ID NO: 3 - DNA, 33 bp, Artificial Sequence
  Synthetic primer
cgcggatccttattatgcagatttgttccaaac

SEQ ID NO: 4 - DNA, 1230 bp, Pseudomonas putida
  misc_feature: Gene Sequence for Putative Alanine Racemase from Pseudomonas putida KT2440; NCBI Accession number NC_002947.3
atgccctttcgccgtacccttctggctgcatccctggcacttctgatcaccggacaggcccccctgtatgcggcaccaccgttgtcgatggacaacggcaccaacaccctgaccgtgcaaaacagcaatgcctgggtcgaagtcagcgccagcgccctgcagcacaacatccgcacgctgcaggccgagctggccggcaagtccaagctgtgcgccgtgctcaaggccgatgcctatggccacggtatcggcctggtaatgccatcgatcatcgcccaaggcgtgccctgcgtggcggtggccagcaacgaggaggcccgcgtggtccgcgccagtggcttcaccgggcaactggtgcgggtacgcctggccagcctcagcgagctggaagatggcttgcagtacgacatggaagagctggtgggcagcgcggaatttgcccgccaggccgatgccatcgccgcgcgccatggcaagaccttgcgcattcacatggcgctcaactccagcggcatgagccgcaacggggtggagatggccacctggtccggccgtggcgaagcgctgcagatcaccgaccagaagcacctcaagctggtcgcgctgatgacccacttcgccgtggaagacaaggacgatgtacgcaagggcctggcggcattcaacgagcagaccgactggttgatcaagcacgccaggctggaccgcagcaagctcaccctgcacgccgccaactcgttcgctacgctggaagtgccggaagcgcgcctggacatggtacgaacgggtggcgcgctgttcggcgacaccgtgccggcgcgcaccgagtacaaacgtgcgatgcagttcaaatcgcacgtggcggcggtgcacagctatccggccggcaacaccgtgggctatgaccgcaccttcaccctggcccgtgattcgcggctggccaacattacggtcgggtactccgatggctaccgccgggtattcaccaacaagggccatgtgctgatcaacggccaccgtgtgccggtcgtgggcaaggtgtcgatgaacacgctgatggtcgatgtcaccgacttccctgatgtgaaggggggtaacgaagtggtgctgttcggcaagcaggccgggggcgaaatcacccaggccgagatggaagaaatcaacggcgcgttgctcgccgatttgtacaccgtatggggcaattccaacccgaagatactcgtcgactga

SEQ ID NO: 5 - DNA, 27 bp, Artificial Sequence
  Synthetic primer
aatccatatgccctttcgccgtaccct

SEQ ID NO: 6 - DNA, 27 bp, Artificial Sequence
  Synthetic primer
cgcggatcctcagtcgacgagtatctt

SEQ ID NO: 7 - protein, 409 aa, Pseudomonas putida
  misc_feature: Amino Acid Sequence for Putative Alanine Racemase from Pseudomonas putida KT2440; NCBI Accession number NP_745855.1
MPFRRTLLAASLALLITGQAPLYAAPPLSMDNGTNTLTVQNSNAWVEVSASALQHNIRTLQAELAGKSKLCAVLKADAYGHGIGLVMPSIIAQGVPCVAVASNEEARVVRASGFTGQLVRVRLASLSELEDGLQYDMEELVGSAEFARQADAIAARHGKTLRIHMALNSSGMSRNGVEMATWSGRGEALQITDQKHLKLVALMTHFAVEDKDDVRKGLAAFNEQTDWLIKHARLDRSKLTLHAANSFATLEVPEARLDMVRTGGALFGDTVPARTEYKRAMQFKSHVAAVHSYPAGNTVGYDRTFTLARDSRLANITVGYSDGYRRVFTNKGHVLINGHRVPVVGKVSMNTLMVDVTDFPDVKGGNEVVLFGKQAGGEITQAEMEEINGALLADLYTVWGNSNPKILVD

SEQ ID NO: 8 - protein, 698 aa, Taxus canadensis
  misc_feature: Amino Acid Sequence for Phenylalanine Aminomutase from Taxus canadensis; NCBI Accession number AAT47186
MGFAVESRSHVKDILGLINTFNEVKKITVDGTTPITVAHVAALARRHDVKVALEAEQCRARVETCSSWVQRKAEDGADIYGVTTGFGACSSRRTNQLSELQESLIRCLLAGVFTKGCASSVDELPATATRSAMLLRLNSFTYGCSGIRWEVMEALEKLLNSNVSPKVPLRGSVSASGDLIPLAYIAGLLIGKPSVVARIGDDVEVPAPEALSRVGLRPFKLQAKEGLALVNGTSFATALASTVMYDANVLLLLVETLCGMFCEVIFGREEFAHPLIHKVKPHPGQIESAELLEWLLRSSPFQDLSREYYSIDKLKKPKQDRYALRSSPQWLAPLVQTIRDATTTVETEVNSANDNPIIDHANDRALHGANFQGSAVGFYMDYVRIAVAGLGKLLFAQFTELMIEYYSNGLPGNLSLGPDLSVDYGLKGLDIAMAAYSSELQYLANPVTTHVHSAEQHNQDINSLALISARKTEEALDILKLMIASHLTAMCQAVDLRQLEEALVKVVENVVSTLADECGLPNDTKARLLYVAKAVPVYTYLESPCDPTLPLLLGLEQSCFGSILALHKKDGIETDTLVDRLAEFEKRLSDRLENEMTAVRVLYEKKGHKTADNNDALVRIQGSRFLPFYRFVREELDTGVMSARREQTPQEDVQKVFDAIADGRITVPLLHCLQGFLGQPNGCANGVESFQSVWNKSA

SEQ ID NO: 9 - DNA, 2103 bp, Taxus canadensis
  misc_feature: Wild Type Aminomutase seq (Walker) Taxus canadensis; NCBI Acc# AY582743
atggggtttgccgtggaatcgcgttctcacgtaaaggatatattggggctgatcaacacgttcaacgaggtgaagaaaattacagtagacggtacgacccccatcacggtggcccatgtcgcggcgctggcccggaggcatgacgtgaaggttgcgttggaggcggagcaatgcagagcccgtgtggaaacctgctcttcgtgggtgcagcgcaaggcggaagacggcgccgacatatacggcgtcaccacgggctttggcgcgtgctcgagccgtaggaccaaccagctgagcgagctgcaggagtcgcttatacgctgcctgctcgcgggggtgtttactaaaggatgcgcttcctccgtcgacgagctccctgcgaccgccacccgcagcgccatgctgctccgccttaatagttttacctatggatgttccggcatccggtgggaggtcatggaagcgctggaaaagcttctcaacagcaatgtctctcctaaagtgcctctccgaggatctgtgagcgcttcgggagacctcatcccgctcgcctacattgcagggctcctgattgggaagcctagcgtagtcgctcgcataggcgacgatgtcgaggtccctgcgcccgaagcgttgagcagggtggggctgcggccattcaagctccaggccaaagaagggctggcgctcgtcaacggcacctccttcgccaccgcgctcgcctccaccgtcatgtacgacgccaatgttctgttgctgctcgtcgaaacgctttgcggaatgttctgcgaggtgatctttggaagggaggagttcgcgcatccgctgatccataaagtgaagccgcacccaggccagatcgaatcggcggagctgctcgagtggctgctgcggtcgagcccgtttcaggacctgtcgagggagtattacagtattgataagctgaagaaaccgaaacaggatcgctatgctctgaggtcgagcccgcagtggttggctcctctggtgcagacaatcagagacgccaccactacagtggagacggaggtcaattccgccaatgataaccccatcattgaccacgccaatgacagggctctccatggtgcgaatttccagggcagcgccgtcggcttctacatggactacgtgcgcatcgcagtcgccgggctggggaaactcttgttcgctcagttcacggagctgatgatcgaatattacagcaacggcctaccggggaacctctccctggggccggacctgagcgtggactacggcctcaaggggctcgacatcgccatggccgcctacagctccgagcttcagtacctggcgaatcccgtgaccacacacgtgcacagcgcggaacagcacaaccaggacatcaactctctggcgctcatctccgcccgcaagacggaggaagcgttggatatcttaaagctcatgatcgcctcgcatttaacagcaatgtgccaggcggtggaccttcggcagctcgaagaagccctagtaaaagtcgtggagaatgtcgtttccacccttgcagacgaatgcggcctccctaacgacacaaaggcgaggcttttatatgtagccaaagcggtgcctgtttacacatacctggaatccccctgcgaccctacgcttcccctcttgttaggcctggaacagtcctgtttcggttccattctggctctccacaaaaaagacggcattgagacggacaccttggtcgatcggctcgccgagttcgagaagcggctgtccgaccgcctggaaaacgagatgacggcagtgagggttttgtacgaaaagaaagggcataaaactgcagacaacaacgacgccctcgtgagaatccagggttccagattccttcctttttacagatttgttcgggaagagctcgacacaggtgtgatgagtgcgagaagagagcagacgccgcaagaggacgtgcagaaagtgttcgatgcaattgccgacggcagaattacggtgcctctgctgcactgcctgcaagggtttctcggccaaccaaatgggtgcgccaacggcgtcgagtcgttccaaagtgtttggaacaaatctgcgtgaaagggc

SEQ ID NO: 10 - DNA, 2277 bp, Taxus chinensis
  misc_feature: Aminomutase seq (Steele) Fig2 Taxus chinensis; NCBI Acc# AY724735
gctctcatatggggtttgccgtggaatcgcgttctcacgtaaaggatatattggggctgatcaacgcgttcaacgaggtgaagaaaattacagtagacggtacgacccccatcacggtggcccatgtcgcggcgctggcccggaggcatgacgtgaaggttgcgttggaggcggagcaatgcagagcccgtgtggaaacctgctcttcgtgggtgcagcgcaaggcggaagacggcgccgacatatacggcgtcaccacgggcttcggcgcgtgctcgagccggaggaccaaccggctgagcgagctgcaggagtcgctcatacgctgcctgctcgcgggggtgtttactaaaggatgcgctccctccgtcgacgagctccccgcgaccgccacccgcagcgccatgctgctccgccttaatagttttacctatggatgttccggcatccggtgggaggtcatggaagcgctggaaaagcttctcaacagcaatgtctctcctaaagtgcctctccggggttctgtgagcgcttcgggagacctcatcccgctcgcctacattgcagggctcctgatcgggaagcctagcgtaatcgctcgcataggcgacgatgtcgaggtccctgcgcccgaggcgttgagcagggtggggcttcggccattcaagctccaggccaaagaagggctggcgctcgtcaacggcacctccttcgccaccgcggtcgcctccaccgtcatgtacgacgccaatgttctgttgctgctcgtcgaaacgctttgcggaatgttctgcgaggtgatctttggaagggaggagttcgcgcatccgctgatccataaagtgaagccgcacccgggccagatcgaatcggcggagctgctcgagtggctgctgcggtcgagcccgtttcaggagctgtcgagggagtattacagtattgataagctgaagaaaccgaaacaggatcgctatgctctgaggtcgagcccgcagtggttggctcctctggtgcagacaatcagagacgccaccactacagtggagacggaggtcaattccgccaatgataaccccatcattgaccacgccaatgacagggctctccatggtgcgaatttccagggcagcgccgtcggtttctacatggactacgtgcgcatcgcagtagccgggctggggaaactcttgttcgctcagttcacggagctgatgatcgaatattacagcaacggcctaccggggaacctctccctggggccggacctgagcgtggactacggcctcaaggggctcgacatcgccatggccgcctacagctccgagcttcagtacctggcgaatcccgtgaccacacacgtgcacagcgcggaacagcacaaccaggacatcaactctctggcgctcatctccgcccgcaagacggaggaggcgttggatatcttaaagctcatgatcgcctcgcatttaacagcaatgtgccaggcggtggaccttcggcagctcgaagaagccctagtaaaagtcgtggagaatgtcgtttccacccttgcagacgaatgcggcctccctaacgacacaaaggcgaggcttttatatgtagccaaagcggtgcctgtttacacatacctggaatccccctgcgaccccacgcttcccctcttgttaggcctgaaacagtcctgtttcgataccattctggctctccacaaaaaagacggcattgagacggacaccttggtcgatcggctcgccgagttcgagaagcggctgtccgaccgcctggaaaacgagatgacggcagtgagggttttgtacgaaaagaaagggcataaaacggcagacaacaacgacgccctcgtgagaatccagggttccaaattccttcctttttacagatttgttcgggaagagctcgacacaggtgtgatgagtgcgagaagagagcagacgccgcaagaggacgtgcagaaagtgttcgatgcaattgccgacggcagaattacggtgcctctactgcactgcctgcaagggtttctcggccaaccaaatgggtgcgccaacggcgtctagtcgttccaaagtgtttggaacaaatctgcgtgatttctgcgtgaatatttgagtagaatttcagattgttcggttcgtgtgatgtttgcagtagaaattccgcagaagcgactgtagctttgcgagaattgttagtttgtgagtgaaatttatctgattggcttcctatgtaaaccctaattaatttttgttttaaaaggatccc

SEQ ID NO: 11 - DNA, 2411 bp, Taxus chinensis
  misc_feature: Aminomutase seq (Steele) Fig4 Taxus chinensis; NCBI Acc# AY724736
ttcagttttatctcgctcaagtttcaatcttttaattttaaagttattttccttgctctgcgatggggtttgccgtggaatcgcgttctcacgtaaaggatatattggggctgatcaacgcgttcaacgaggtgaagaaaattacagtagacggtacgacccccatcacggtggcccatgtcgcggcgctggcccggaggcatgacgtgaaggttgcgttggaggcggagcaatgcagagcccgtgtggaaacctgctcttcgtgggtgcagcgcaaggcggaagacggcgccgacatatacggcgtcaccacgggcttcggcgcgtgctcgagccggaggaccaaccagctgagcgagctgcaggagtcgctcatacgctgcctgctcgcgggggtgtttactaaaggatgcgcttcctccgtcgacgagctccccgcgaccgccacccgcagcgccatgctgctccgccttaatagttttacctatggatgttccggcatccggtgggaggtcatggaagcgctggaaaagcttctcaacagcaatgtctctcctaaagtgcctctccggggttctgtgagcgcttcgggagacctcatcccgctcgcctacattgcagggctcctgatcgggaagcctagcgtaatcgctcgcataggcgacgatgtcgaggtccctgcgcccgaggcgttgagcagggtggggcttcggccattcaagctccaggccaaagaagggctggcgctcgtcaacggcacctccttcgccaccgcggtcgcctccaccgtcatgtacgacgccaatgttctgttgctgctcgtcgaaacgctttgcggaatgttctgcgaggtgatctttggaagggaggagttcgcgcatccgctgatccataaagtgaagccgcacccgggccagatcgaatcggcggagctgctcgagtggctgctgcggtcgagcccgtttcaggagctgtcgagggagtattacagtattgataagctgaagaaaccgaaacaggatcgctatgctctgaggtcgagcccgcagtggttggctcctctggtgcagacaatcagagacgccaccactacagtggagacggaggtcaattccgccaatgataaccccatcattgaccacgccaatgacaggtaatgtatatcattcgtcgttaagcaatctgccgacttcatagagattccaaaacttctgacgaaaaagtggataagacggggctcctagaaagttttccttttaaagatgaactatatttttttattacggactagatttcgacggttttgtccgatccattggcagggctctccatggtgcgaatttccagggcagcgccgtcggtttctacatggactacgtgcgcatcgcagtcgccgggctggggaaactcttgttcgctcagttcacggagctgatgatcgaatattacagcaacggcctaccggggaacctctccctggggccggacctgagcgtggactacggcctcaaggggctcgacatcgccatggccgcctacagctccgagcttcagtacctggcgaatcccgtgaccacacacgtgcacagcgcggaacagcacaaccaggacatcaactctctggcgctcatctccgcccgcaagacggaggaggcgttggatatcttaaagctcatgatcgcctcgcatttaacagcaatgtgccaggcggtggaccttcggcagctcgaagaagccctagtaaaagtcgtggagaatgtcgtttccacccttgcagacgaatgcggcctccctaacgacacaaaggcgaggcttttatatgtagccaaagcggtgcctgtttacacatacctggaatccccctgcgaccccacgcttcccctcttgttaggcctgaaacagtcctgtttcgataccattctggctctccacaaaaaagacggcattgagacggacaccttggtcgatcggctcgccgagttcgagaagcggctgtccgaccgcctggaaaacgagatgacggcagtgagggttttgtacgaaaagaaagggcataaaacggcagacaacaacgacgccctcgtaagaatccagggttccaaattccttcctttttacagatttgttcgggaagagctcgacacaggtgtgatgagtgcgagaagagagcagacgccgcaagaggacgtgcagaaagtgttcgatgcaattgccgacggcagaattacggtgcctctgctgcactgcctgcaagggtttctcggccaaccaaatgggtgcgccaacggcgtctagtcgttccaaagtgtttggaacaaatctgcgtgatttctgcgtgaatatttcagtagaatttcagattgttcggttcgtgtgatgtttgcagtagaaattccgcagaagcgactgta

SEQ ID NO: 12 - DNA, 2233 bp, Taxus x media
  misc_feature: Aminomutase seq (Steele) Fig5 Taxus x media; NCBI Acc# AY724737
atggggtttgccgtggaatcgcgttctcacgtaaaggatatattggggctgatcaacacgttcaacgaggtgaagaaaattacagtagacggtacgacccccatcacggtggcccatgtcgcggcgctggcccggaggcatgacgtgaaggttgcgttggaggcggagcaatgcagagcccgtgtggaaacctgctcttcgtgggtgcaacgcaaggcggaagacggcgccgacatatacggcgtcaccacgggcttcggcgcgtgctcgagccggaggaccaaccagctgagcgagctgcaggagtcgctcatacgctgcctgctcgcgggggtgtttactaaaggatgcgcttcctccgtcgacgagctccccgcgaccgtcacccgcagcgccatgctgctccgccttaatagttttacctatggatgttccggcatccggtgggaggtcatggaagcgctggaaaagcttctcaacagcaatgtctctcctaaagtgcctctccgaggatctgtgagcgcttcgggagacctcatcccgctcgcctacattgcagggctcctgatcgggaaacctagcgtaatcgctcgcataggcgacgatgtcgaggtccctgcgcccgaggcgttgagcagggtggggctgcggccattcaagctccaggccaaagaagggctggcgctcgtcaacggcacctccttcgccaccgcgctcgcctccaccgtcatgtacgacgccaatgttctgttgctgctcgtcgaaacgctttgcggaatgttctgcgaggtgatctttggaagggaggagttcgcgcatccgctgatccataaagtgaagccgcacccgggccagatcgaatcggcggagctgctcgagtggctgctgcggtcgagcccgtttcaggagctgtcgagggagtattacagtattgataagctgaagaaaccgaaacaggatcgctatgctctgaggtcgagcccgcagtggttggctcctctggtgcagacaatcagagacgccaccactacagtggagacggaggtcaattccgccaatgataaccccatcattgaccacgccaatgacaggtaatgtacatcattcgtcgttaagcaatctgccgacttcatagagattccaaaacttctgacaaaaaagtggataagatggggctcctagaaagttttccttttaaagatgaactatatttttttataactgactagatttcgctggttttgtccgatccattggcagggctctccatggtgcgaatttccagggcagcgccgtcggcttctacatggactacgtgcgcatcgcagtcgccgggctggggaaactcttgttcgctcagttcacggagctgatgatcgaatattacagcaacggcctaccggggaacctctccctggggccggacctgagcgtggactacggcctcaaggggctcgacatcgccatggccgcctacagctccgagcttcagtacctggcgaatcccgtgaccacacacgtgcacagcgcggaacagcacaaccaggacatcaactctctggcgctcatctccgcccgcaagacggatgaggcgttggatatcttaaagctcatgatcgcctcgcatttaacagcaatgtgccaggcggtggaccttcggcagctcgaagaagccctagtaaaagtcgtggagaatgtcgtttccacccttgcagacgaatgcggcctccctaacgacacaaaggcgaggcttttatatgtagccaaagcggtgcctgtttacacatacctggaatcccccagcgaccccacgcttcccctcttgttaggcctgaaacaatcctgtttcgattccattctggctctccacaaaaaagacggaattgagacggacaccttggtcgatcggctcgccgagttcgagaagcggctgtccgaccgcctggaaaacgagatgacggcagtgagggttttgtacgaaaagaaagggcataaaacggcagacaacaacgacgccctcgtgagaatccagggttccaaattccttcctttttacagatttgttcgggacgagctcgacacaggtgtgatgagtgcgagaagagagcagacgccgcaagaggacgtgcagaaagtgttcgatgcaattgccgacggcagaattacggtgcctctgctgcactgcctgcaagggtttctcggccaaccaaatgggtgcgccaacggcgtctag

SEQ ID NO: 13 - DNA, 2235 bp, Taxus canadensis
  misc_feature: Aminomutase seq (Steele) Fig6 Taxus canadensis; NCBI Acc# AY724738
atggggtttgccgtggaatcgcgttctcacgtaaaggatatattggggctgatcaacacgttcaacgaggtgaagaaaattacagtagacggtacgacccccatcacggtggcccatgtcgcggcgctggcccggaggcatgacgtgaaggttgcgttggaggcggagcaatgcagagcccgtgtggaaacctgctcttcgtgggtgcagcgcaaggcggaagacggcgccgacatatacggcgtcaccacgggctttggcgcgtgctcgagccggaggaccaaccagctgagcgagctgcaggagtcgcttatacgctgcctgctcgcgggggtgtttactaaaggatgcgcttcctccgtcgacgagctccctgcgaccgtcacccgcagcgccatgctgctccgccttaatagttttacctatggatgttccggcatccggtgggaggtcatggaagcgctggaaaagcttctcaacagcaatgtctctcctaaagtgcctctccgaggatctgtgagcgcttcgggagacctcatcccgctcgcctacattgcagggctcctgattgggaagcctagcgtaatcgctcgcataggcgacgatgtcgaggtccctgcgcccgaggcgttgagcagggtggggctgcggccattcaagctccaggccaaagaagggctggcgctcgtcaacggcacctccttcgccaccgcgctcgcctccaccgtcatgtacgacgccaatgttctgttgctgctagtcgaaacgctttgcggaatgttctgcgaggtgatctttggaagggaggagttcgcgcatccgctgatccataaagtgaagccgcacccaggccagatcgaatcggcggagctgctcgagtggctgctgcggtcgagcccgtttcaggacctgtcgagggagtattacagtattgataagctgaagaaaccgaaacaggatcgctatgctctgaggtcgagcccgcagtggttggctcctctggtgcagacaatcagagacgccaccactacagtggagacggaggtcaattccgccaatgataaccccatcattgaccacgccaatgacaggtaatgcatatcattcgtcgttaagcaatctgccgacttcatagagattccaaaacttctgacaaaaaagtggataagatggggctcctagaaagttttccttttaaagatgaactatatttttttataactgactagatttcgctggttttgtccgatccattggcagggctctccatggtgcgaatttccagggcagcgccgtcggcttctacatggactacgtgcgcatcgcagtcgccgggctggggaaactcttgttcgctcagttcacggagctgatgatcgaatattacagcaacggcctaccggggaacctctccctggggccggacctgagcgtggactacggcctcaaggggctcgacatcgccatggccgcctacagctccgagcttcagtacctggcgaatcccgtgaccacacacgtgcacagcgcggaacagcacaaccaggacatcaactctctggcgctcatctccgcccgcaagacggatgaggcgttggatatcttaaagctcatgatcgcctcgcatttaacagcaatgtgccaggcggtggaccttcggcagctcgaagaagccctagtaaaagtcgtggagaatgtcgtttccacccttgcagacgaatgcggcctccctaacgacacaaaggcgaggcttttatatgtagccaaagcggtgcctgtttacacatacctggaatccccctgcgaccctacgcttcccctcttgttaggcctgaaacagtcctgtttcgattccattctggctctccacaaaaaagacggcattgagacggacaccttggtggatcggctcgccgagttcgagaagcggctgtccgaccgcctggaaaacgagatgacggcagtgagggttttgtacgaaaagaaagggcataaaactgcagacaacaacgacgccctcgtgagaatccagggttccaaattccttcctttttacagatttgttcgggacgagctcgacacaggtgtgatgagtgcgagaagagagcagacgccgcaagaggacgtgcagaaagtgttcgatgcaattgccgacggcagaattacggtgcctctgctgcactgcctgcaagggtttctcggccaaccaaatgggtgcgccaacggcgtctagac

SEQ ID NO: 14 - protein, 687 aa, Taxus chinensis
  misc_feature: Aminomutase Protein Seq (Steele) seq Fig Taxus chinensis; NCBI Acc# AY724735
MGFAVESRSHVKDILGLINAFNEVKKITVDGTTPITVAHVAALARRHDVKVALEAEQCRARVETCSSWVQRKAEDGADIYGVTTGFGACSSRRTNRLSELQESLIRCLLAGVFTKGCAPSVDELPATATRSAMLLRLNSFTYGCSGIRWEVMEALEKLLNSNVSPKVPLRGSVSASGDLIPLAYIAGLLIGKPSVIARIGDDVEVPAPEALSRVGLRPFKLQAKEGLALVNGTSFATAVASTVMYDANVLLLLVETLCGMFCEVIFGREEFAHPLIHKVKPHPGQIESAELLEWLLRSSPFQELSREYYSIDKLKKPKQDRYALRSSPQWLAPLVQTIRDATTTVETEVNSANDNPIIDHANDRALHGANFQGSAVGFYMDYVRIAVAGLGKLLFAQFTELMIEYYSNGLPGNLSLGPDLSVDYGLKGLDIAMAAYSSELQYLANPVTTHVHSAEQHNQDINSLALISARKTEEALDILKLMIASHLTAMCQAVDLRQLEEALVKVVENVVSTLADECGLPNDTKARLLYVAKAVPVYTYLESPCDPTLPLLLGLKQSCFDTILALHKKDGIETDTLVDRLAEFEKRLSDRLENEMTAVRVLYEKKGHKTADNNDALVRIQGSKFLPFYRFVREELDTGVMSARREQTPQEDVQKVFDAIADGRITVPLLHCLQGFLGQPNGCANGV